(19) United States
(12) Patent Application Publication
(10) Pub. No.: US 2024/0304320 A1
(43) Pub. Date: Sep. 12, 2024

(54) SURGICAL SYSTEMS AND CONTROL METHODS

(71) Applicant: Arthrex, Inc., Naples, FL (US)

(72) Inventors: Gregory St. Clair, Santa Barbara, CA (US); Ken Olbrish, Santa Barbara, CA (US); Lee Holoien, Santa Barbara, CA (US); Craig Speier, Santa Barbara, CA (US); Tim Jackson, Pittsburgh, PA (US)

(73) Assignee: Arthrex, Inc., Naples, FL (US)

(21) Appl. No.: 18/601,238

(22) Filed: Mar. 11, 2024

Related U.S. Application Data
(60) Provisional application No. 63/451,007, filed on Mar. 9, 2023.

(51) Int. Cl. G16H 40/63; G06F 13/36

(52) U.S. Cl. CPC G16H 40/63; G06F 13/36; G06F 2213/40

(57) ABSTRACT

A surgical control system is in communication with a plurality of surgical devices. In operation, the surgical devices broadcast device data including messages indicative of at least one of a control state and a detected condition associated with the operation of each of the plurality of surgical devices via a communication bus in communication with a plurality of device controllers of the plurality of surgical devices. Image data is captured demonstrating a surgical site and a surgery status is identified in response to the image data. Aggregate data is generated, including the surgery status and the device data, in a packet. The aggregate data is communicated over the communication bus.

### CROSS-REFERENCE TO RELATED APPLICATIONS

This application claims priority under 35 U.S.C. § 119(e) and the benefit of U.S. Provisional Application No. 63/451,007 entitled SURGICAL SYSTEMS AND CONTROL METHODS, filed on Mar. 9, 2023, by St. Clair, et al., the entire disclosure of which is incorporated herein by reference.

### BACKGROUND

The present disclosure generally relates to a control system for surgical devices and, more particularly, to a distributed control system and communication interface configured to communicate device data for automated or assisted operations. Modern surgical suites may incorporate a wide variety of medical or surgical devices that may be utilized to assist in surgical procedures, monitor patient health statistics and vital signs, as well as control various lighting, ventilation, sterilization, and additional devices associated with a medical suite. In various implementations, the disclosure provides for systems and methods that may assist in the operation of such devices by improving the interoperability of medical or surgical devices used in various combinations.

### SUMMARY

The disclosure provides for a surgical control system that may facilitate the distributed communication of device data including control states and detected conditions associated with the operation of a plurality of medical or surgical devices. In various implementations, the device data may be communicated via a device network or communication bus, over which each of the plurality of surgical devices may broadcast or report updates. The updates may be communicated at standard intervals and/or in response to changes to the corresponding control states and/or detected conditions. The device data may be accessed by a plurality of device controllers corresponding to each of the plurality of surgical devices. In operation, the device controllers may independently initiate one or more automated control settings or control prompts based on the programming or control structure associated with a particular surgical device. In this way, the disclosure may provide for improved operation of a variety of interconnected surgical or medical devices via a distributed control platform.

In some implementations, the control system may include a camera apparatus, which may be in the form of an endoscopic, arthroscopic device, surgical cameras, or similar imaging devices in communication with the plurality of medical devices. In operation, the camera apparatus may be implemented to capture image data demonstrating a surgical site, and, based on the image data, a control unit or system controller of the camera apparatus may identify a surgery status of the surgical site. Such an identification may include the determination of a wide variety of patient conditions, procedural steps, surgical device conditions, surgical site conditions, or various characteristics that may be identified from the image data captured by the camera apparatus. In addition to the image data, the camera apparatus may further interpret the device data associated with each of the plurality of surgical devices communicated over the device network to further improve the interpretation of the image data to determine the surgery or surgical site status.

In some implementations, the system controller of the camera apparatus may access and generate aggregate data in a packet. Each packet of aggregate data may comprise the surgery status, the device data, and various control or status data associated with the plurality of surgical devices. The aggregate data may be arranged or formatted in a standard that is readily accessed and processed by the device controllers of the surgical devices over the device network. In this way, the surgical control system may provide for the plurality of surgical devices, including the camera apparatus, to automatically respond to the device data, surgery status, and/or the aggregate data or, in some cases, provide proposed control settings to medical professionals to assist in the operation of the surgical control system.

In various implementations, the comprehensive operation of the system controller may provide for the improved operation and control of an inflow and/or outflow of fluid to an operating site as controlled by a surgical pump. In operation, a system controller may be configured to identify a blood metric indicating a level of blood in the surgical site and a visibility metric of the image data that is independent of the blood metric. In response to the blood metric and the visibility metric, the system controller may identify a control setting for the surgical pump. Additionally, the control setting or settings for the surgical pump may differ based on the blood level identified by the blood metric even under circumstances where the visibility metric identifies similar visibility results in the image data. Accordingly, the blood and visibility metrics may be monitored and applied individually or in combination to adjust the control settings of the surgical pump.

These and other features, objects and advantages of the present disclosure will become apparent upon reading the following description thereof together with reference to the accompanying drawings.

### BRIEF DESCRIPTION OF THE DRAWINGS

FIG. 1 is a pictorial diagram of a surgical control system demonstrating an exemplary operation of a control routine for distributed control;

FIG. 2 is a schematic diagram of a surgical control system for a plurality of surgical devices;

FIG. 3 is a simplified block diagram demonstrating a device network for a surgical control system;

FIG. 4 is a process diagram demonstrating an exemplary control routine of a device network for a surgical control system;

FIG. 5A is a flow chart demonstrating a method for distributed control of a plurality of surgical devices;

FIG. 5B is a continued flow chart from FIG. 5A demonstrating a distributed control method for a plurality of surgical devices;

FIG. 6 is an illustrative process diagram demonstrating a lavage process associated with the operation of a surgical pump;

FIG. 7 is a graphic representation of a vision processing method for a surgical control system;

FIG. 8 is a graphic representation of an exemplary vision processing method;

FIG. 9A is a graphic representation of an exemplary vision processing method;

FIG. 9B is a process diagram demonstrating the vision processing method of FIG. 9A;

FIG. 10 is a flow chart demonstrating a method for operating a vision processing module for a surgical control system;

FIG. 11 is a plot demonstrating an exemplary process for tracking operating properties associated with a surgical pump;

FIG. 12 is a representative diagram of a user interface for a surgical control system;

FIG. 13 is an illustrative process diagram demonstrating various use cases of a surgical control system in connection with a plurality of surgical devices; and

FIG. 14 is a flow chart demonstrating a method for controlling the operation of a surgical device based on a patient condition or user preference.

### DETAILED DESCRIPTION

In the following description of the preferred implementations, reference is made to the accompanying drawings, which show specific implementations that may be practiced. Wherever possible, the same reference numbers will be used throughout the drawings to refer to the same or like parts. It is to be understood that other implementations may be utilized and structural and functional changes may be made without departing from the scope of this disclosure.

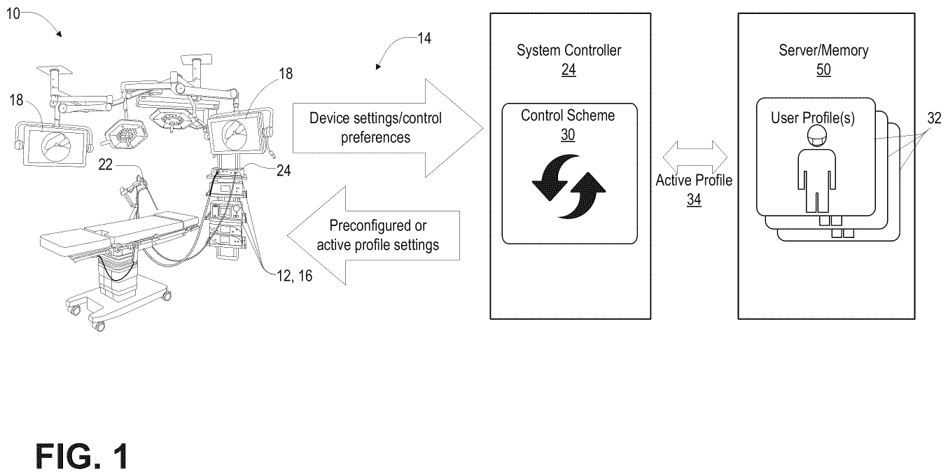

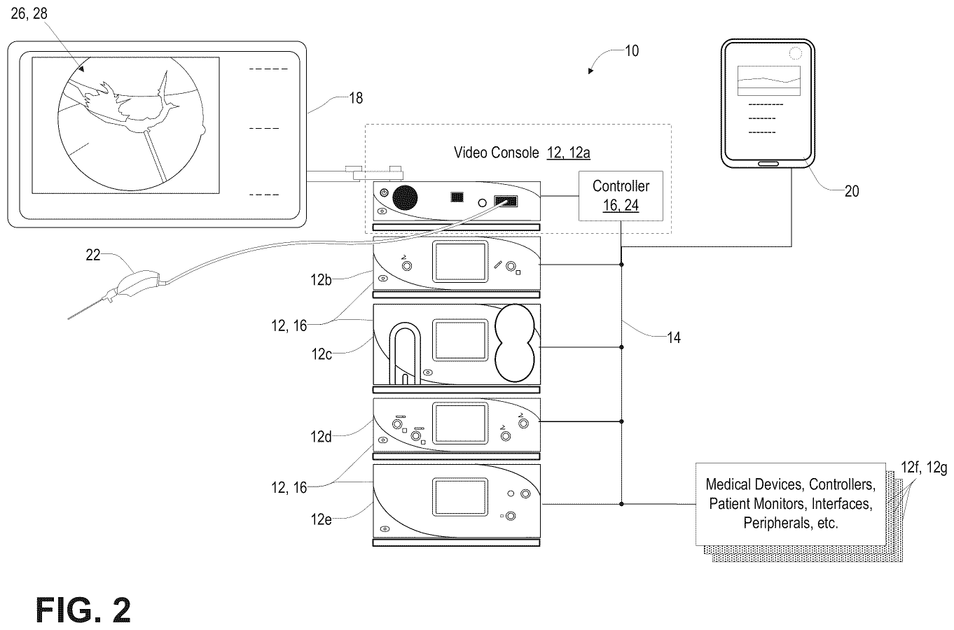

Referring to FIGS. 1 and 2, exemplary diagrams of a control system 10 for a plurality of surgical devices 12 are shown. As illustrated in FIG. 2, the surgical devices 12 are communicatively coupled via a device network 14. In various implementations, each of the surgical devices 12 may incorporate or be in communication with a corresponding device controller 16. As demonstrated in FIG. 1, each of the device controllers 16 may be incorporated as integral components of the corresponding surgical devices 12. However, in some implementations, one or more of the surgical devices 12 may share a common device controller 16. In this configuration, each of the surgical devices 12 may report and receive device data via the device network 14 in the form of messages or packets indicative of one or more control states or detected conditions associated with the operation of each of the surgical devices 12. The communication of the device data among the device controllers 16 of the surgical devices 12 may provide for the independent control of the operation of each of the plurality of surgical devices 12 to facilitate the coordinated operation of the control system 10.

In the exemplary implementation shown, the surgical devices 12 may include a video console 12a, an electrosurgical console 12b, an arthroscopy pump controller 12c, a resection console 12d, an insufflation console 12e, and additional devices 12f. As shown, the devices 12f or consoles may include a variety of surgical or medical devices, including anesthesia control devices, infusion pump controllers, patient monitors, controls or interface peripherals, tourniquet or restraint controls, and/or various medical or surgical devices. Accordingly, though specific devices are discussed and demonstrated in various examples presented in the application, the scope of the application shall not be limited to the examples provided.

In addition to the medical or surgical devices 12, various computerized devices, including displays 18 and tablets or computers 20, may be communicatively connected to the device network 14 via a wired or wireless communication interface. As shown in FIG. 1, the display 18 may be associated with the video console 12a and may present image data 26 captured by a camera apparatus 22 exemplified as an endoscope or arthroscope. In operation, a system controller 24 (e.g., a camera control unit [CCU]) may be implemented as the device controller 16 associated with the video console 12a. The system controller 24 may serve as a common communication hub of the control system 10 and may process and aggregate communications to and from each of the surgical devices 12 on the device network 14. As later discussed in reference to specific examples, the video console 12a may be configured to process the image data 26 from the camera apparatus 22 to identify a surgery status or characteristics of a surgical site via a vision-based identification technique, which may correspond to computer vision in the form of object detection, image classification, sequence classification, convolutional neural network classification, spectral recognition, etc. The surgery status may be incorporated in aggregate data from each of the surgical devices 12 and reported by the system controller 24 over the device network 14.

The surgery status identified by the system controller 24 (e.g., the CCU) may include patient conditions, procedural steps, surgical device conditions, site conditions, or various characteristics that may be identified based on the image data 26 captured by the camera apparatus 22. For example, if the video console 12a identifies that increased blood is present at the surgical site from monitoring the image data 26, the corresponding surgery status may be reported over the device network 14 via the system controller 24. In this configuration, each of the device controllers 16 of the surgical devices 12 may adjust their operations responsive to the increased blood detection or any other device data reported via the device network 14. In the specific example of the blood detection, the arthroscopy pump controller 12c may respond by increasing a fluid circulation rate to and from the surgical site to clear and improve the visibility of the surgical site within the image data 26 as depicted on the display 18. Further, if the video console 12a recognizes a clear field of view within the surgical site from monitoring the image data 26, the arthroscopy pump controller 12c may respond by adjusting the operation by sequentially decreasing pump pressure, in this instance to the pre-set pressure minimum previously determined. In this way, the system 10 may provide for the distribution of the device data or state information as well as the vision-based surgery status to each of the surgical devices 12, such that control of the system 10 may be coordinated and independently implemented by each of the device controllers 16.

In addition to processing the image data 26 to determine the vision-based surgery status, the system controller 24 may further be configured to receive, process, and aggregate the device data reported by each of the surgical devices 12. With the device data combined, the system controller 24 may report the combined device data as aggregate data back to the device network 14. In some implementations, the aggregate data reported by the system controller 24 may include the surgery status or visually detected characteristics of the surgical site 28 with the aggregate data. In cases where the aggregate data includes the vision-based surgery status, the device controllers 16 of the surgical devices 12 may monitor and respond by adjusting one or more settings accordingly to the vision-based condition associated with the surgery status reported in the aggregate data. Accordingly, the device controllers 16 of the surgical devices 12 may control various settings or prompt a user to approve proposed settings based on the aggregate data.

Still referring to FIGS. 1 and 2, in some implementations, the device data reported by the surgical devices 12 may be accessed by the system controller 24 or CCU to assist or be processed as a factor in the determination of the vision-based status or surgery status. For example, the system controller 24 may utilize the device data to inform or filter potential statuses or procedures identified in the image data 26 with the computer vision. In operation, the device data may be processed by the system controller 24 in combination with the image data 26 to categorize or filter a number of potential vision states or modify a status based on the device data. In this way, the system 10 may process the device data accessed over the device network 14 to assist or improve the determination of the surgery status or computer vision assessment of the image data. As further discussed in the following detailed examples, the device data may be processed by one or more processors or controllers of the video console 12a to filter a plurality of potential surgery statuses associated with the condition of the surgical site 28. In this way, the control system 10 may provide for the coordinated operation of each of the connected surgical devices 12 while also maintaining independent or distributed control of each of the devices 12 as provided by the device controllers 16.

Referring specifically to FIG. 1, the system 10 may provide for customized or user-specific control schemes 30 or preferences that may be detected and defined for a plurality of users and accessed via a corresponding user profile 32. The control schemes 30 may be attributed to the user profiles 32 via manual programming or machine learning algorithms, sometimes referred to as artificial intelligence. For example, in response to the detection of the device data and/or the surgery status, the system controller 24 may identify one or more user-specific control settings or control prompts associated with an active profile 34 of the user profiles 32. The identification of such user-specific control settings or control prompts may be detected by the system controller 24 and stored in a server or memory 50. The control settings for each user profile 32 may be identified in response to inputs to each of the surgical devices 12 via corresponding interfaces, the computer or tablet 20, and/or various peripherals or inputs to the system 10.

Throughout operation, the system controller 24 may detect control preferences as combinations of the device data and/or the surgery status reported via the device network 14. These control preferences may be stored to the control scheme 30 and associated with the user profile 32, such that the system controller 24 may learn the user preferences associated with each of the surgical devices 12 over time. In this way, the system 10 may be programmed or learn the control preferences and provide for customized control of the each of the surgical devices 12. As discussed herein, the control scheme 30 may include various control settings including display settings, device settings, user input or control mapping preferences, as well as various automated control prompts or settings that may be initiated in response to the device data, surgery status, and/or the aggregate data.

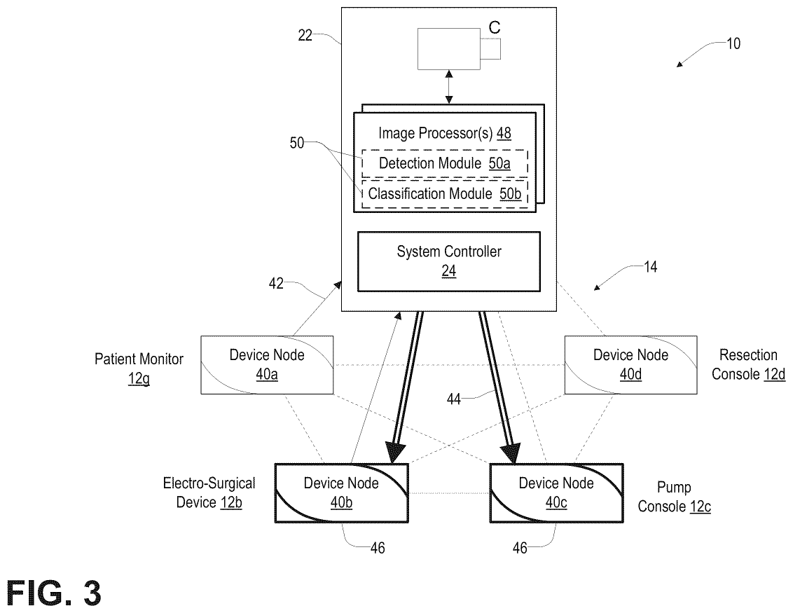

Referring now to FIG. 3, a schematic diagram of the device network 14 of the control system 10 is shown demonstrating an example of a distributed control implementation of the system 10. As previously discussed, each of the surgical devices 12 may be in communication with a corresponding device controller 16. In operation, each of the device controllers 16 may report the device data associated with the current operation or control state of the corresponding surgical device 12. For example, the electrosurgical console 12b may report a control state in the form of a current electrosurgical treatment setting and update the setting via one or more messages communicated over the device network 14 throughout the operation of the electrosurgical console 12b. Additionally, the device controller 16 of the electrosurgical console 12b may report one or more detected or monitored conditions associated with the operation of the electrosurgical console 12b. For example, in some implementations, the electrosurgical device associated with or controlled by the electrosurgical console 12b may include one or more sensors (e.g., temperature sensors, pressure sensors, etc.) or feedback devices (e.g., current sensors, load sensors, fault sensors, etc.), the status of which may be monitored and reported by the corresponding device controller 16 as messages over the device network 14. Such communication may similarly be provided by the corresponding device controllers 16 of each of the surgical devices 12 in communication with the device network 14. In this way, the device controllers 16 may distribute the device data associated with the operation of each of the surgical devices 12, such that the device data 42 is accessible for the independent control of the surgical devices 12 by the corresponding device controllers 16. As previously discussed, the device data 42 may also be combined with the surgery status or image-based status information into aggregate data 44 by the system controller 24 of the video console 12a.

Still referring to diagram shown in FIG. 3, the device network 14 is demonstrated among a plurality of exemplary surgical devices 12 referred to as device nodes 40. Each of the device nodes 40 is connected via broken lines that represent the shared communication of the device data 42 and aggregate data 44 communicated among the plurality of corresponding surgical devices 12 linked to the device nodes 40. Accordingly, the device data 42 reported by each of the device controllers 16 of the surgical devices 12 may be accessible via the communication lines represented by the dashed lines shown in FIG. 3. Additionally, the aggregate data 44 may be broadcast from the system controller 24 to each of the device controllers 16 as represented by the dashed lines. Though the communication of the device data 42 is discussed in reference to specific surgical devices 12 and nodes 40, the device data 42 and aggregate data 44 may be broadcast over the device network 14, such that each of the devices 12 connected may access and independently respond according to the messages and/or packets received.

In addition to the general communication among the surgical devices 12 as provided by the device network 14, the diagram shown in FIG. 3 further demonstrates a plurality of arrows representing device data 42 and aggregate data 44 that is communicated to all of the connected devices but further processed and implemented by some of the surgical devices 12 to control or update the corresponding operation. The devices that act responsive to the device data 42 and aggregate data 44 are distinguished from the remaining surgical devices 12 in FIG. 3 by the arrows associated with the device data 42 and aggregate data 44 communicated to the corresponding or responsive surgical devices 46. To be clear, the responsive surgical devices 46 represent a subset of the surgical devices 12 demonstrated in connection with the device network 14 to illustrate that each of the surgical devices 12 may be programmed to independently respond to the device data 42 and aggregate data 44 available to all of the devices 12 over the device network 14.

In the example shown, the device nodes 40 correspond to a first device node 40a, a second device node 40b, a third device node 40c, and a fourth device node 40d. The system controller 24 of the video console 12a may respond to communications from the first device node 40a, which may correspond to patient data associated with the blood pressure, heart rate, blood/oxygen level, or various other characteristics that may be monitored via the patient monitor 12g. Additionally, the device data 42 may be reported from the second device node 40b, which may correspond to a device setting or ablation intensity of the electrosurgical console 12b. In response to the device data 42 reported by the first node 40a and the second node 40b, the system controller 24 may generate and report the aggregate data 44 to each of the device nodes 40 and corresponding surgical devices 12. In addition to the device data 42, the system controller 24 may further supplement or modify the aggregate data 44 based on a vision-based condition or surgery status detected in the image data as previously discussed. Accordingly, the aggregate data 44 may be combined, modified, and distributed by the system controller 24 to each of the device nodes 40 in communication via the device network 14. Though discussed in various examples in reference to the system controller 24, the aggregate data 44 may be combined and processed by one or more additional or alternative device controllers 16 associated with the surgical devices 12 in general.

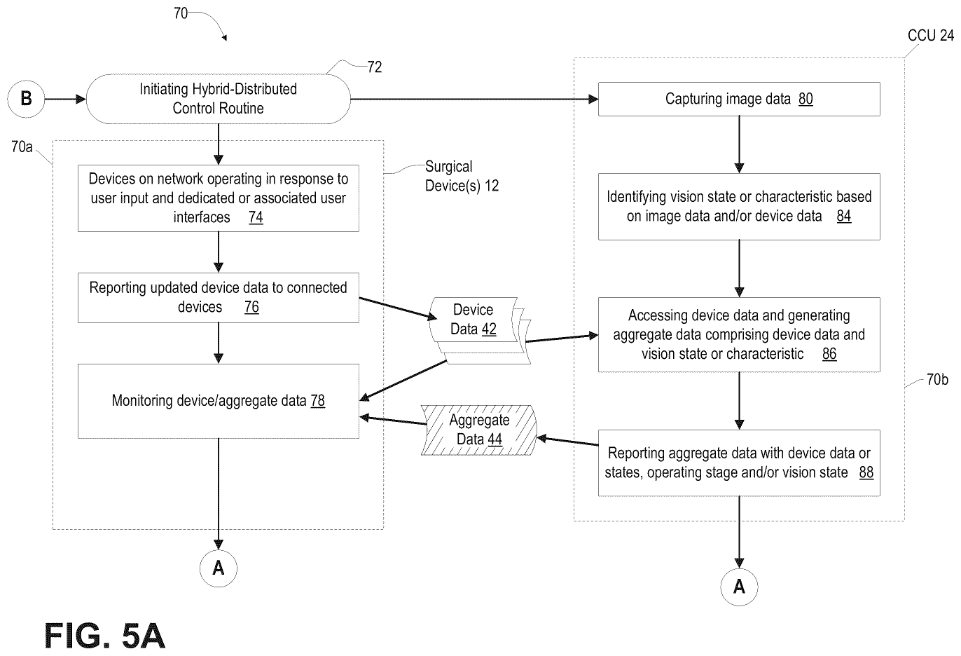

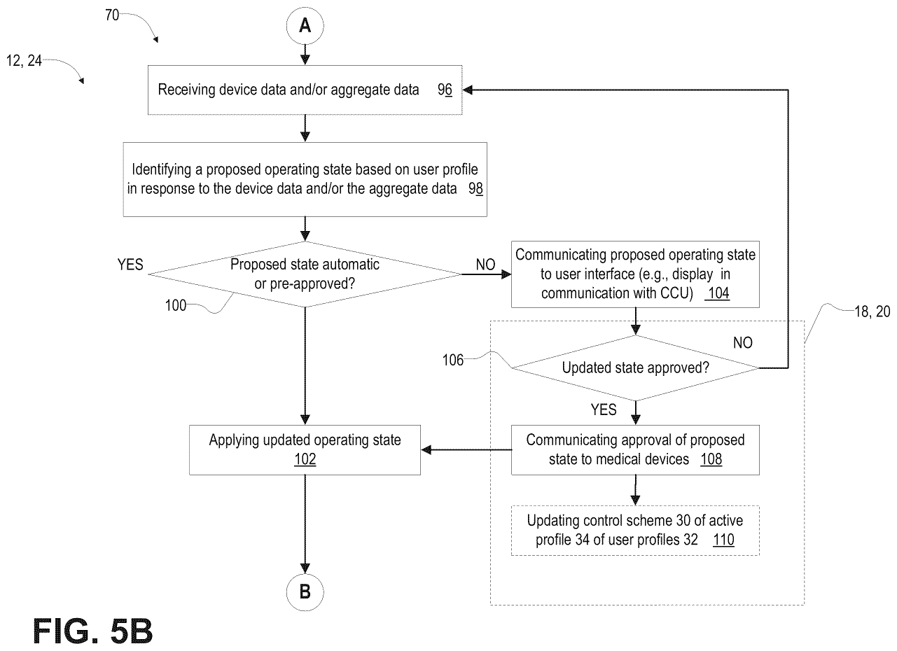

Still referring to FIG. 3, some of the responsive surgical devices 46 represented by the second device node 40b and the third device node 40c may adjust one or more operational settings responsive to the device data 42 and/or the aggregate data 44. As shown, the electrosurgical console 12b associated with the second node 40b may respond to the aggregate data 44 by adjusting an ablation setting or ablation intensity. Additionally, or alternatively, as later discussed in the example shown in FIG. 5B, the electrosurgical console 12b or other surgical devices 12 associated with the system 10 may identify and communicate a proposed control setting. A proposed control setting may be presented to a user of the system 10 to request an update to the operation of one or more of the surgical devices 12 rather than providing automated control. Accordingly, the system 10 may provide for semi-automated control of a variety of settings or control instructions for the surgical devices 12.

Referring again to FIG. 3, in addition to controlling the electrosurgical console 12b, the arthroscopy pump controller 12c associated with the third node 40c may respond to the aggregate data 44 by adjusting a pressure, flow rate, or circulation rate of the fluid circulated through the surgical site 28 (e.g., a joint space or patient cavity). In addition to the responsive behavior illustrated by the devices associated with each of the second node 40b and the third node 40c, the patient monitor associated with the first node 40a and, for example, a resection console 12d associated with the fourth device node 40d may not respond or propose a control response to the device data 42 or aggregate data 44 reported over the device network 14. Accordingly, the control system 10 may provide for reporting of the device data 42 among each of the surgical devices 12, such that the device controllers 16 of the surgical devices 12 may process the device data 42 and aggregate data 44 to independently respond to the reported device and/or vision-based surgery status conditions accessed via the device network 14.

Still referring to FIG. 3, the schematic diagram of the video console 12a demonstrates a connection to the camera apparatus 22 as well as the implementation of one or more image processors 48 that may be implemented to facilitate the computer vision techniques discussed herein. For example, the image processors may include one or more digital processing devices including, for example, a central processing unit (CPU) with one or more processing cores, a graphics processing unit (GPU), digital signal processors (DSPs), field programmable gate arrays (FPGAs), application specific integrated circuits (ASICs), and the like. In some configurations, multiple processing devices are combined into a System on a Chip (SoC) configuration while in other configurations the processing devices may correspond to discrete components. In operation, the processor(s) 48 may execute program instructions stored in a memory 50 to perform the operations described herein. As shown, the memory 50 is referenced as a plurality of modules representative of the corresponding control routines stored therein.

In operation, the one or more processor(s) 48 may review or process the image data 26 according to routines established by one or more computer vision routines exemplified as a detection module 50a and a classification module 50b. Accordingly, one or more computer vision routines may be implemented by the processor 48 according to a variety of image processing techniques following the acquisition of the image data 26, which may include preprocessing, filtering, segmentation, and feature extraction, as well as a variety of additional processes that may be instructed by the detection module 50a. In some examples, features identified by the detection module 50a may be interpreted by the classification module 50b as identifiable features or characteristics of the image data 26 to generate or output associated classifications or descriptions referred to herein as the reported vision-based conditions. In some cases, a detection module 50a may be omitted and the vision-based conditions may be identified in response to one or more characteristics of the image data 26 that may be inferred based on the operation of the classification module 50b or similar vision-based detection or classification routines as discussed herein. The vision-based conditions may include the surgery status, stage or step of a procedure, or characteristics of the surgical site 28 as previously discussed. In this way, the system controller 24 may provide for the application of computer vision techniques to modify the aggregate data 44 reported to the surgical devices 12 and improve the coordinated operation of the control system 10 responsive not only to the device data 42 but also to current or anticipated conditions at the surgical site 28, such as potential bleeding events.

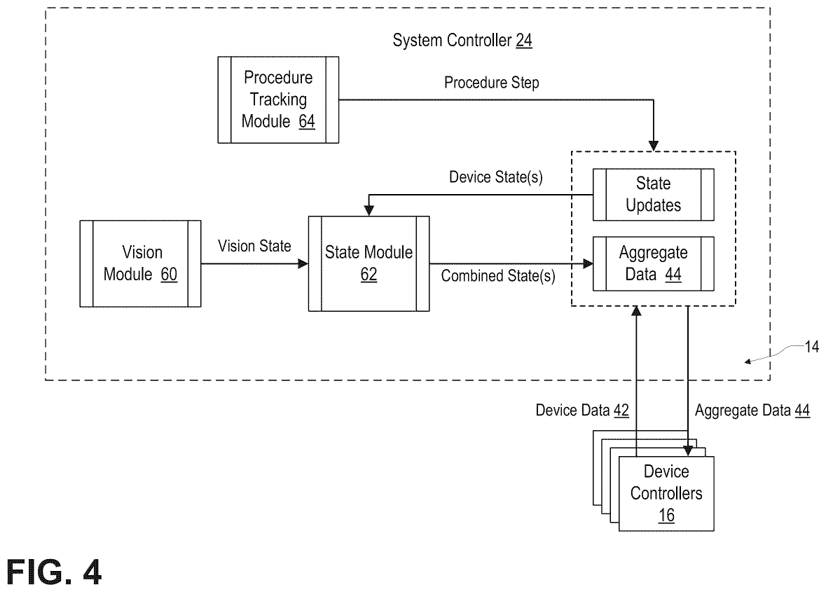

Referring now to FIG. 4, a process diagram demonstrates an exemplary communication architecture and operating procedure for the control system 10. As shown, the detection module 50a and the classification module 50b may generally be referred to as a vision module 60. In the example shown, the vision state identified by the vision module 60 may serve as an input to a state module 62 or state machine configured to determine and track various combined states of the surgical devices 12 based on the communications received from each of the corresponding device controllers 16. Throughout operation, each of the device controllers 16 may report individual device data 42, including operating states, settings, and/or measured operating values, over the device network 14. The device data 42 may then be accessible over the device network 14 by each of the device controllers 16, as well as the system controller 24 or camera control unit (CCU). In this configuration, the control system 10 may provide for the distributed control of each of the surgical devices 12 in communication with the device network 14.

In response to receipt of the device data 42, the system controller 24 may generate the aggregate data 44 by processing and packaging the combined state information, including settings, operating states, and vision states, as the aggregate data 44 reported over the device network 14. In addition to the device data 42, the system controller 24 may additionally include a procedure tracking module 64 that may track and communicate the current step of a predetermined sequence of steps operating associated with a specific surgical procedure to the state module 62. As provided in further detail in various examples in the following description, the combination of the vision state as well as one or more of the device states and/or setting of each of the surgical devices 12 may allow the state module 62 to make various inferences regarding proposed operating settings to be automatically activated or proposed for activation for one or more of the surgical devices 12 of the control system 10. Such inferred and/or contextual information may be reported back to the device network 14 as the aggregate data 44 and provide meaningful input to each of the surgical devices 12, allowing for intuitive operating suggestions and/or automated controls to improve the operation of the system 10.

FIGS. 5A and 5B demonstrate an exemplary control routine or method 70 providing for the independent control of each of the surgical devices 12 over the device network 14. As shown, the method 70 may be referred to as a distributed control routine based on the independent nature of the control of each of the surgical devices 12 via the device controllers 16. As previously discussed, the aggregate data 44 may be used alone or in combination with the device data 42. In the specific example shown, the method steps associated with the surgical devices 12 are demonstrated in the left column 70a and the steps associated with the system controller 24 are demonstrated in the right column 70b. However, it shall be understood that the steps associated with the surgical device 12 in the first column 70a may similarly be implemented by system controller 24. Accordingly, the method 70 may be adjusted to suit nearly any number of surgical devices and also may be implemented with or without the video console 12a in cases where the tasks associated with the system controller 24 do not include image capture operations.

Referring to FIG. 5A, the method 70 may generally be initiated in response to the activation of one or more of the surgical devices 12 of the control system 10 (72). Following the activation, the surgical devices 12 may be manually or automatically controlled by one or more users to provide their conventional applications (i.e., pumping fluid, delivering electrosurgical energy, measuring patient vitals or sensor data, etc.) (74). In some cases, the operation of the surgical devices 12 may be controlled by one or more external devices, for example, the computer or tablet 20, via communication over the device network 14. However, generally, the surgical devices 12 may be configured to respond to inputs to each of their dedicated or associated user interfaces, which may comprise one or more foot pedals, switches, remote controls, touchscreens, etc. Accordingly, the method 70 may provide a combination of manual device operation with suggested or automated control updates depending on the specific application.

Following step 74, based on the operation of each of the surgical devices 12, each of the associated device controllers 16 may report device data 42 to each of the surgical devices 12 connected over the device network 14 (76). Following or concurrent to the reporting of the device data 42, the device controllers 16 may further monitor device data 42 and/or aggregate data 44 reported by the other surgical devices 12 communicated over the device network 14 (78). Following step 78, the device controllers 16 of each of the surgical devices 12 may continue to subroutine A, which is continued in FIG. 5B. Before moving on to discuss the exemplary operation of the system controller 24 or camera control unit, it is important to note that the timing of the reporting and monitoring of the device data 42 associated with each of the surgical devices 12 may vary. To be clear, the frequency as well as the associated timing of each instance of the reporting and monitoring described in reference to steps 76, 78, and later in reference to step 88 may vary from one device to another. Such operation may assist in facilitating the independent control of each of the surgical devices 12 without necessitating temporally coordinated control of the surgical devices 12 with a central controller.

Concurrent to steps 74-78 as discussed in reference to the surgical devices 12, steps 80-88 may be processed by the system controller 24. In operation, the method 70 as associated with the video console 12a may begin in response to the capturing and/or processing of the image data 26 (80). In general, the system controller 24 (e.g., the CCU) may process the image data to identify the surgery status or various characteristics associated with the surgical site 28 (84). Throughout the capture of the image data and identification of the vision-based condition of the surgical site 28, the system controller 24 may further access the device data 42 reported by the surgical devices 12 and generate the aggregate data 44 comprising the device data 42 as well as one or more vision-based conditions via the device network 14 (86). Once the aggregate data 44 is generated, the data 44 may be reported or broadcast over the device network 14 (88). In some cases, a characteristic or vision state of the surgical site 28 may additionally be reported in the aggregate data 44 in cases where such information is available and has been processed by the CCU or system controller. Following step 88, the system controller 24 may also continue to subroutine A, which is continued in FIG. 5B.

Referring now to FIG. 5B, the method steps associated with any of the surgical devices 12, including the video console 12a and the associated system controller 24, may proceed through subroutine A and return to the method demonstrated in FIG. 5A via reference path B. As shown, the controllers 16, 24 of the surgical devices 12 may each similarly receive or selectively access the device data 42 and/or the aggregate data 44 in step 96. Based on the device data 42 and/or aggregate data 44, each of the controllers 16, 24 may identify a proposed or automated operating state independently based on the corresponding programming associated with each of the surgical devices 12 (98). As previously discussed, the proposed or automated operating state of the surgical devices 12 may be identified based on an active profile 34 of the user profiles 32. Accordingly, the proposed state noted in step 100 may be identified in response to a previously identified user preference associated with a combination of the device data 42 and/or the surgery status communicated over the device network 14 by the surgical devices 12 including the video console 12a.

Based on the active profile 34 and the corresponding programming or control instructions, each of the controllers 16, 24 may identify a proposed operating state and may also determine if the proposed operating state associated with the device data 42 and/or aggregate data 44 is an automatic control state, pre-approved, or a manual state in step 100. In cases where the proposed state is automatically accepted and updated by the controller 16, 24 of the surgical device 12, the proposed operating state may be applied in step 102. If the proposed state requires a confirmation, each of the corresponding controllers 16, 24 associated with the surgical devices 12 may respond according to their associated programming or control instructions to continue to step 104 to request manual authorization or approval. If manual approval is required, the method 70 may continue to step 104 and communicate the proposed operating state to a user interface (e.g., the display 18, the computer or tablet 20, etc.) and present a prompt requesting authorization of the proposed state. If the proposed state is approved in step 106, the method 70 may continue to step 108 and communicate the approval of the proposed state via the device network 14 where the corresponding controller 16, 24 may access the approval as a message. Once the approval is communicated via the message over the device network 14, the associated controller 16, 24 may apply the proposed operating state in step 102 as previously discussed. Following step 102, the method 70 may follow reference path B and continue to operate following steps 74-78 and 80-88 as previously discussed.

In addition to communicating the approval for the proposed operating state to the associated controller 16, 24 in step 108, the method 70 may further communicate an update to the control scheme 30 of the active profile 34 as previously discussed in reference to FIG. 1 (110). Over time, such updates to the control scheme 30 may be implemented by the system 10 to provide a customized user experience that is associated with each of the user profiles 32. As a result, the control settings and configurations for each of the surgical devices 12 may be proposed by the system 10 and utilized to populate the corresponding settings associated with the user profiles 32. Additionally, the system may provide for advanced learning operations that not only automatically detect conditions associated with the device data and the aggregate data but further generate custom programming of settings as well as the operations of the surgical devices 12 responsive to the control preferences recorded for each of the users as described in step 106. In this way, the system 10 may automatically update the control schemes 30 associated with the operation of the surgical devices 12 for each of the user profiles 32.

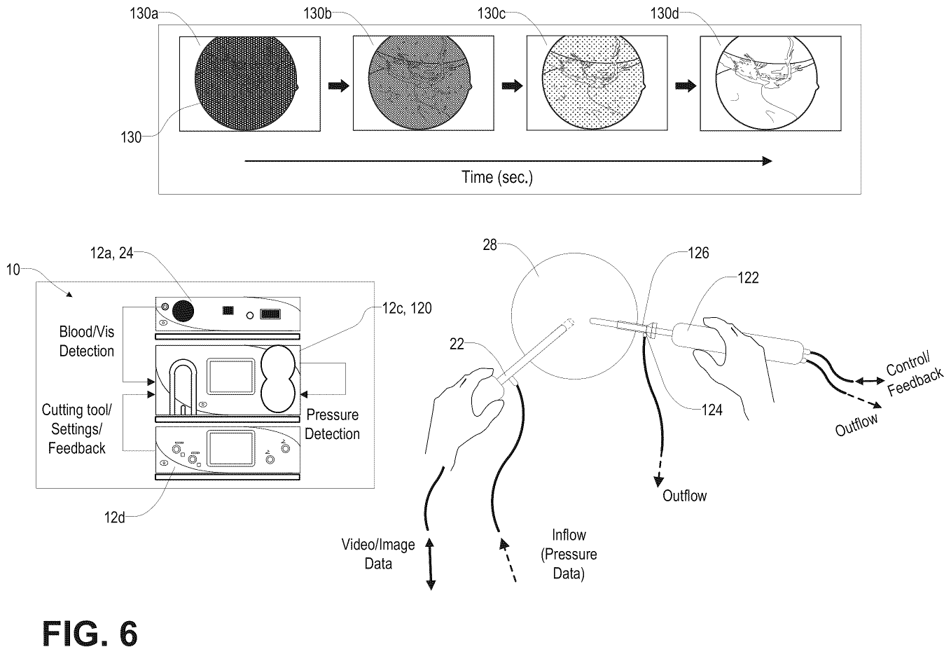

Referring now to FIG. 6, exemplary operation of the pump controller 12c of a surgical pump 120 is discussed in reference to a bleeding event detected by the vision module 60 of the system controller 24. The bleeding event may be the result of the operation of a resection tool 122 (e.g., a shaver, rasp, drill, burr, etc.) applied at the surgical site 28 representing a patient cavity. In the example shown, the pump 120 may be utilized to control an inflow of surgical fluid by monitoring the supply pressure or local pressure at the surgical site 28. For example, in some implementations, the surgical pump 120 may include an inflow pump, which may be operated in combination with an outflow pump to control the flow rate and/or pressure. The inflow may be supplied via a cannula of the camera apparatus 22, and an outflow of the fluid may be recovered from the surgical site 28 via a lumen of the cutting or resection tool 122 as well as an outflow port 124 of a cannula 126. In cases where only an inflow pump is included in the surgical pump 120, the fluid exchange or flow through the surgical site 28 or cavity may be the result of the pressure differential between the surgical site 28 and the outflow port 124. In such cases, the flow rate may be indirectly controlled. In other cases, the surgical pump 120 may include both inflow and outflow control pumps or devices. Accordingly, the pump controller 12c may control the inflow and/or the outflow to and from the surgical site 28 to maintain hemostasis of the patient cavity ensure that the corresponding anatomy is effectively displayed in a field of view 130 of the camera apparatus 22.

Throughout surgical procedures similar to that exemplified, various operations may result in decreased visibility in the field of view 130 represented by the exemplary images 130a-130d. For example, the operation of the cutting tool 122 or resection device may result in particulate matter (e.g., tissue, bone fragments, etc.) within the patient cavity. Additionally, the removal of tissue, drilling, incisions, or various surgical tasks may result in bleeding or hemorrhaging that may also impact the visibility. As shown in the first image 130a, the surgical site 28 is represented in a low-visibility state that may correspond to a bleeding event resulting from a resection operation. As shown, the first image 130a may include particulate matter and decreased visibility resulting from the introduction of blood into the surgical fluid in the patient cavity. In response to the detection of such a low-visibility condition by the module 60, the system controller 24 may respond by activating a lavage or rinsing technique to adjust the pressure and/or fluid exchange rate and remove the particulate material, blood, and/or tissues from the patient cavity.

In the example shown, the vision module 60 may identify the low-visibility state as having a visibility below a visibility threshold Tᵥ of a visibility metric and being classified as a blood-positive detection impacting the clarity of the image data 26. In such an example, the vision module 60 may process the image data 26 to identify that bleeding is likely occurring at the surgical site 28. In response to the detection of the potential bleeding event, the system controller 24 may activate or prompt a user of the system 10 to activate a lavage routine. As later discussed in further detail, the prompt may be accompanied with a conditional mapping of one or more user inputs on a user interface of the camera apparatus 22 or various connected devices 12 to receive a confirmation input to accept the proposed activation. In each case, responsive to the automated or user-activated lavage routine, the pump controller 12c may activate an increase in surgical fluid provided to the inflow via the pump 120 to increase the pressure and tamponade the bleeding at the surgical site 28. Additionally, in some implementations, the outflow communicated through the outflow port 124 of the cannula 126 may be regulated to control the inflow pressure and simultaneously rinse the blood and/or particulate matter from the surgical site 28. As demonstrated by the sequence of images 130a-130d, the lavage routine may result in the control of the bleeding by increasing the fluid pressure while also rinsing the patient cavity. In this way, the system 10 may clear the field of view 130 of the camera apparatus 22 from blood and debris to ensure that surgical procedures can be completed effectively.

As discussed herein, the image data 26 may correspond to still images, a series of image frames in an image feed, and/or any form of video or visual representation of the field of view 130 of the camera apparatus 22. For example, the image data may correspond to a video feed captured at a frame rate depicting change in the field of view 130 over time. Alternatively, the image data may correspond to images sampled periodically or in response to one or more events or operations associated with the system 10 that may be communicated over the device network 14. Accordingly, the system 10 and corresponding methods may be implemented in a variety of ways to suit a desired application.

In addition to prompting or automatically activating the lavage procedure, the vision module 60 of the system controller 24 may additionally monitor the effectiveness of the lavage or rinsing procedure by monitoring the change in the corresponding image data 26 over time as represented by the images 130a-130d. In response to an improvement in visibility, the vision module 60 may update the vision state to indicate that the bleeding event has been controlled. In operation, the vision module 60 may identify the cessation of the bleeding event as a result of the visibility of the image data improving, as demonstrated in the fourth image 130d, or as a result of corresponding improvement in the visibility as demonstrated in the third image 130c. As a result of the improved visibility detected by the vision module 60, the pump controller 12c may be apprised of the updated operating state of the control system 10 and prompt a user to deactivate the lavage routine and return to a baseline pressure. In this way, the control system 10 may provide for improved patient outcomes and limit the negative impact of extravasation by actively assessing the qualities and/or properties of the image data 26 captured by the camera apparatus 22 and limiting the duration of increased pressure applied at the surgical site 28.

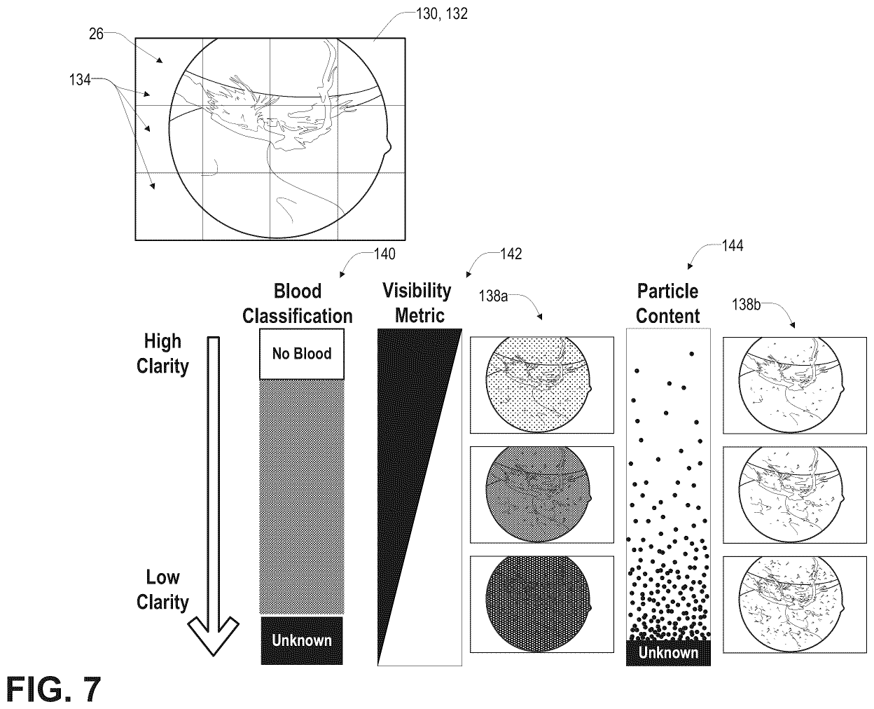

Referring now to FIG. 7, the image data 26 captured by the camera apparatus 22 is demonstrated in various states of visibility or clarity to demonstrate an exemplary vision processing method that may be applied by the vision module 60. Beginning with the representative clear image 132, the vision module 60 may process the image data 26 in a plurality of segments 134 or sectors that may be processed independently to distinguish the relative location of the corresponding visibility conditions within the image data 26. In some implementations, one or more of the segments 134 (e.g., central segments or manually identified segments) may be prioritized as being particularly important to the overall visibility and operation of the camera apparatus 22. Based on the prioritization of the segments 134, the vision module 60 may increase a monitoring frequency of one or more prioritized segments 134 and/or compare the corresponding visibility in the prioritized segments to visibility thresholds requiring higher relative levels of visibility to those segments 134 that are not prioritized and/or centrally located. In this way, the vision module 60 may provide for customized monitoring settings to suit a variety of applications or preferences.

As additionally demonstrated in FIG. 7, the visibility associated with the image data 26 may be classified based on various visually discernable or optically detectable characteristics that may be classified based on one or more metrics or classifications indicating variations from a high level of clarity to a low level of clarity. Exemplary images ranging in clarity associated with the operation of the vision module 60 are demonstrated as images 138a and 138b corresponding to the content demonstrated in the image data 26. In an exemplary embodiment, the vision module 60 may classify the image data independently based on a blood classification of blood classifier 140 and a visibility metric 142. As demonstrated in the first set of images 138a, the images demonstrate decreasing levels of visibility from top to bottom representing changes from a high level of clarity to a low level of clarity as determined by the visibility metric 142. Depending on a predetermined or user-specific visibility threshold Tᵥ, the system 10 may prompt or otherwise control the operation of the pump controller 12c to improve the visibility condition at the surgical site 28.

Though the visibility metric 142 may provide general insight to the system controller 24 and the pump controller 12c as to how to control the inflow and, in some cases, the outflow of the surgical pump 120, the blood classifier 140 may identify specific conditions at surgical site 28 to activate or propose different control strategies for the pump based on the presence of blood. For example, a low visibility associated with a visibility metric 142 may be communicated via the device network 14 to inform the pump controller 12c to increase a fluid exchange or rinse of the surgical site 28. Additionally, a blood-positive classification from the blood classifier 140 may indicate that the decreased visibility of the visibility metric 142 is associated with bleeding at the surgical site 28. Such state information may inform the pump controller 12c that the increased fluid exchange should be accompanied by an increase in fluid pressure provided via the inflow to effectively tamponade the bleeding and clear the image data 26. In this way, the blood classifier 140 and the visibility metric 142 may be monitored by the vision module 60 independently, such that the surgical pump may vary the control settings associated with different events detected in the image data 26.

In operation, the blood classifier 140 of the vision module 60 may detect or classify the image data as blood-positive or blood-negative based on color content or color response associated with the illumination of the surgical site 28 via a range of wavelengths of light. The visibility metric 142 may be measured by the vision module 60 as a relative level of sharpness or contrast associated with the image data 26. In an exemplary embodiment, the visibility metric 142 may be identified based on a visibility regression model compared to ground truth data based on a measure of image quality. For example, a Neural Image Assessment (NIMA) model may be utilized to identify the visibility metric 142 that is trained to prioritize the visibility of the centralized segments 134 over those segments 134 extending about the perimeter of the field of view 130. In this way, the vision module 60 may process the image data 26 in each of the segments 134 to determine a relative level of clarity quantified by the distribution of visibility metric 142. Data used to train the NIMA model to measure the visibility metric 142 may include one or some combination of image quality or color content metrics, including, but not limited to, a sharpness, local standard deviation, local entropy, Mean Standard Contrast Normalized (MSCN), Blind/Referenceless Image Spatial Quality Evaluator (BRISQUE), Naturalness Image Quality Evaluator (NIQE), manually annotated scores, and/or various similar image processing methods. Though the NIMA model is included as an example, it shall be understood that other models or regression models may be similarly applied.

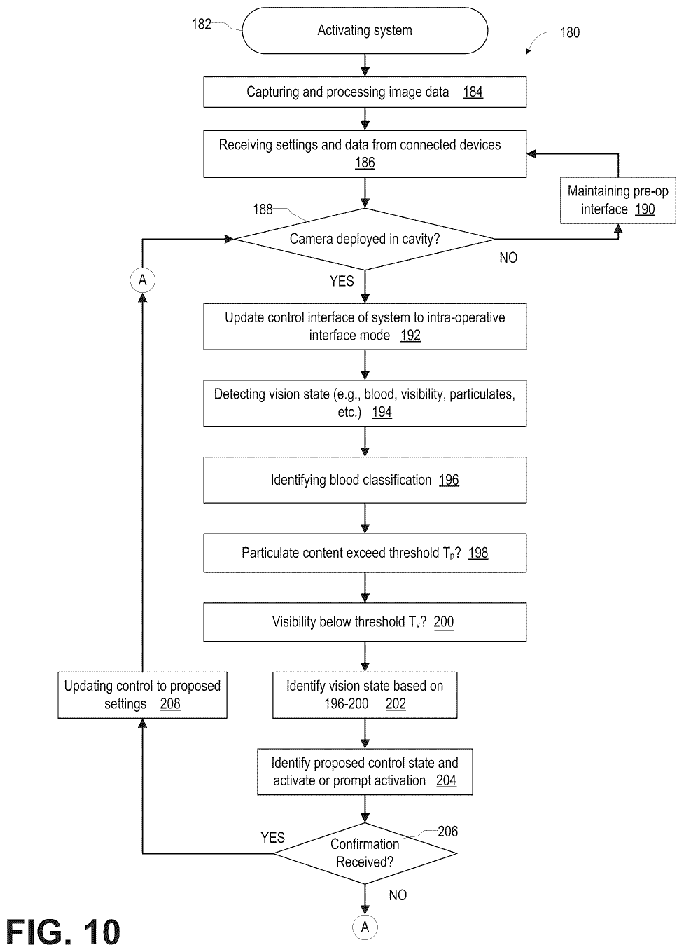

In addition to the blood classifier 140 and visibility metric 142, the vision module 60 may further process the image data 26 to identify various additional discernable characteristics. For example, in some implementations, a particulate or particle-content metric 144 may be identified in the image data 26 to provide further insight to trigger or prompt one or more control settings of the surgical devices 12. For clarity, the visibility metric 142 may quantify various aspects related to the visibility of the image data 26, which may include characteristics also detected by the particle-content metric 144. However, in some cases, it may be beneficial to track the presence of debris or discreet particle content that may alternatively impact the visibility in relation to particle movement and/or intermittent obstructions that may not otherwise be detected within the segments 134 and associated with the visibility metric 142. As shown in the second exemplary image 138b, increasing levels of detected particulate content 168 are shown independent of the underlying turbidity that may consistently cloud the region corresponding to one or more of the segments 134. In operation, the particle-content metric 144 may be identified by the vision module 60 by detecting the movement of one or more discreet objects identified in the image data 26. The identification of discreet or moving particles associated with the particle-content metric 144 may be detected based on various procedures by the vision module 60, including, but not limited to, background subtraction, frame differencing, temporal differencing, optical flow, and various similar image processing techniques. In this way, the vision module 60 may provide additional information to the state module 62, such that the condition associated with the image data 26 in the surgical site 28 may be reported to the surgical devices 12. In particular, the indication of elevated particle content may trigger the pump controller 12c to activate or propose the activation of an inflow and/or outflow fluid adjustment (e.g., increase inflow pressure, increase fluid exchange rate, adjust suction, etc.). In addition to the visibility metric 142 and the particle-content metric 144, additional image processing techniques may be utilized to identify various aspects associated with the operation of the camera apparatus 22, including, but not limited to, determinations of focus of the camera apparatus 22 as well as a location of the camera apparatus 22 (e.g., deployed in the patient cavity or outside of the cavity) as later discussed later in reference to FIG. 10.

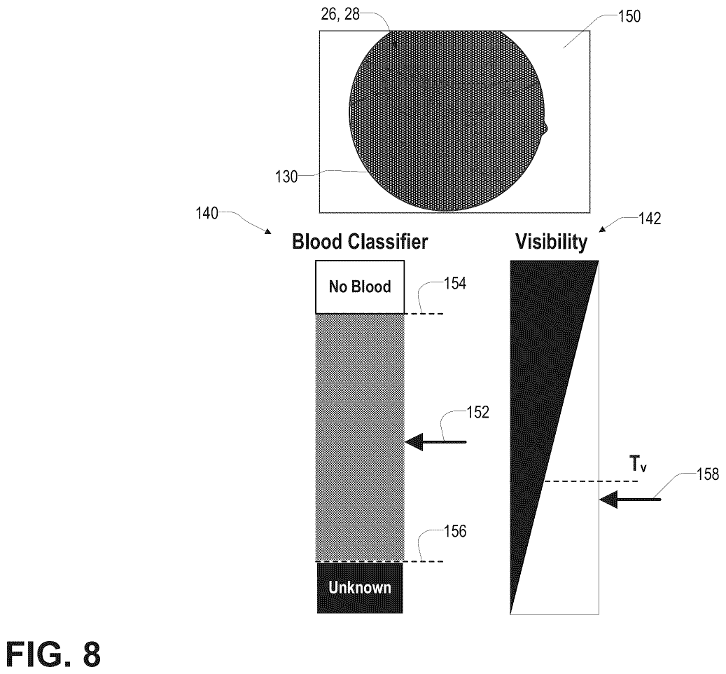

Referring now to FIG. 8, an example of a vision processing method controlled by the vision module 60 is described in reference to an exemplary image 150 of the surgical site 28. In the example shown, the image data 26 includes a blood-positive identification 152 identified by the blood classifier 140 of the vision module 60. The blood-positive identification 152 may be identified by the vision module 60 in response to red color content in the image data 26 in one or more of the segments 134, as previously discussed, exceeding a minimum blood-classification threshold 154. In some cases, the characteristics of the image data 26, including the red content, uniform swirling, or other characteristics, may blend or accumulate beyond a maximum blood-classification threshold 156. Image data 26 exceeding the maximum blood-classification threshold 156 may comprise excess color content or chrominance without adequate luminance to readily classify the condition associated with the field of view 130 as corresponding to the blood-positive condition or identification 152 or other conditions associated with the operation of a camera apparatus 22. For example, unknown blood classifications identified by the blood classifier 140 in excess of a maximum blood-classification threshold 156 may correspond to out-of-focus images, blocked or occluded image sensors, or other visually indeterminant features presented in the image data 26. The detection of such an unknown conditions associated with the blood classifier 140 may be identified by the system controller 24 to output one or more instructions and/or prompts to a user of the control system 10 to indicate that the camera apparatus 22 may be out of focus or otherwise blocked. Accordingly, even conditions exceeding the maximum blood-classification threshold 156 may be identified by the vision module 60 to identify and update the identification of the state of the surgical site 28 as reported by the state module 62. Additionally, though the operation of the blood classifier 140 is primarily described in reference to the color content or chrominance of the image data 26, it shall be understood that color cannot be readily presented due to the limitations associated with the patent publications. Accordingly, the presence of color must be described rather than visually depicted.

Once the vision module 60 determines the blood classification as a blood-positive identification 152, a detected visibility 158 of the exemplary image 150 may further be determined based on the visibility metric 142. In some cases, the detected visibility 158 may be compared to the visibility threshold Tᵥ, which may be preconfigured or associated with a user preference. Such a user preference may be loaded with the active profile 34 corresponding to a user profile 32 as previously discussed. In response to the detected visibility 158 being less then the visibility threshold Tᵥ, the vision module 60 may communicate the state of the surgical site 28 to the state module 62 indicating limited visibility as well as the blood-positive identification 152. The vision state may then be output from the state module 62 and reported in the aggregate data 44 over the device network 14, such that each of the surgical devices 12 may access the reported state and the corresponding device controllers 16 may act responsively to the aggregate data.

As previously discussed, in response to the identification of the detected visibility 158 below the visibility threshold Tᵥ in combination with the blood-positive identification 152, the pump controller 12c may prompt a user of the system 10 to manually activate or otherwise automatically activate a lavage setting of the surgical pump 120. The lavage setting or routine may increase the inflow to the patient cavity causing a resulting pressure increased at the surgical site 28 to slow and control an associated bleeding event. In this way, the system controller 24 may provide for prompts or the automated operation of the surgical pump 120 to ensure that the image data 26 remains clear throughout various procedures.

As discussed in various examples, the activation of one or more proposed settings to control the operation of the surgical pump 120 via the pump controller 12c may require manual activation by a user of the system 10. In various implementations, the activation or confirmation of the proposed control state of one or more of the surgical devices 12 may be presented on the display 18 to prompt the user for confirmation. Additionally, one or more of the device controllers 16 or the system controller 24 may selectively map an input to one of the corresponding user interfaces of the surgical devices 12 to receive a confirmation of the prompted or proposed control setting. For example, in response to a proposed lavage setting identified by the pump controller 12c and/or the system controller 24 as reported over the device network 14, the system controller 24 may selectively map a user input of the camera apparatus 22 to temporarily receive a confirmation of the proposed control setting for the surgical pump 120. A practical example of such an operation may include a prompt on the display 18 requesting that a user of the system 10, “Press camera input 1 to activate lavage.” The user may then engage the corresponding camera input of the camera apparatus 22 to initiate the proposed or prompted lavage process. In this way, the control system 10 may provide for the coordinated operation of the surgical devices 12 by communicating the corresponding states and proposed operational settings over the device network 14 to prompt a user of the system 10 to confirm and activate various settings or operations.

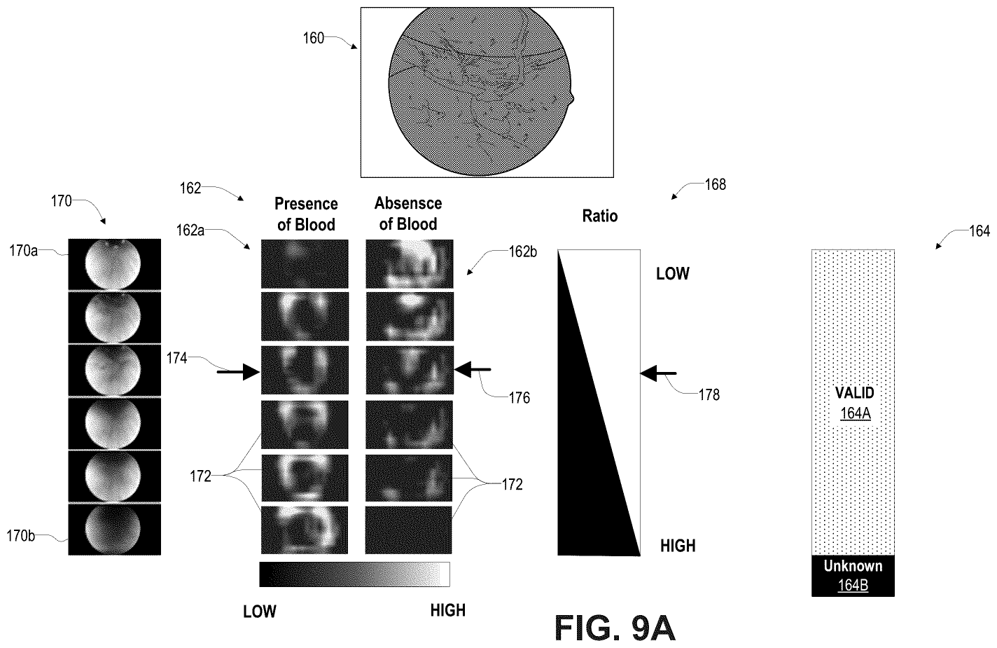

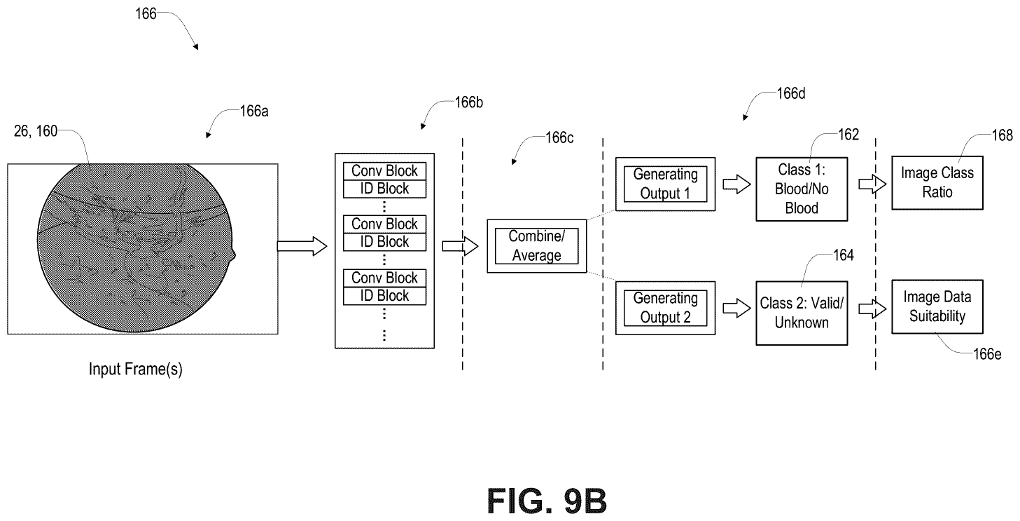

Though discussed as being semi-automated or proposed settings corresponding to the operation of the control system 10, the operations of the pump controller 12c and the system controller 24 may be associated with one or more setting preferences associated with the user profile 32 of the active profile 34. As previously discussed, the user profile 32 of a user may be loaded as the active profile 34 for each procedure associated with the operation of the control system 10. The user profile 32 may include various preferences or operating characteristics set by or trained based on historic operation associated with each corresponding. For example, the user profile 32 may include operational settings for the pump controller 12c to control the surgical pump in reference to conventional operation to maintain hemostasis, the activation of a lavage or rinse routine, as well as preferences associated with the minimum blood-classification threshold 154, the visibility threshold Tᵥ and/or a particulate threshold Tₚ as later discussed in reference to FIG. 9. The control system 10 may prompt each user for one or more preferences and/or learn the preferences associated with the operation of one or more of the surgical devices 12 from historic use. Accordingly, the system 10 may provide for customized operation associated with each of the user profiles 32 to ensure that the proposed operations of the surgical devices are tailored to the needs of each of the specific users.

Though the operation of the surgical pump 120 may vary widely based upon the type of procedure and the preferences of users, an example of a conventional operating pressure required to maintain hemostasis of the patient cavity at the surgical site 28 may be approximately 50 mmHg. Additionally, the increased level of pressure associated with the proposed lavage setting may correspond to a pressure increase ranging from approximately 10% to 50% of the typical operating or baseline pressure. For example, the lavage routine may be processed by the pump controller 12c to control the surgical pump 120 to increase the inflow pressure from 50 mmHg to 70 mmHg. As previously discussed in reference to FIG. 6, the improved clarity of the image data 26 may be monitored by the vision module 60 to determine whether the blood classifier 140, visibility metric 142, and/or the particle-content metric 144 have improved below the corresponding thresholds, each of which may be customized or tailored to the user and stored in the user profile 32. In response to the improved clarity detected in the image data 26, the vision module 60 may update the vision state and communicate the updated state information to the state module 62. The updated state of the fluid at the surgical site 28 may further be reported over the device network 14. In response to the updated state associated with the clarity at the surgical site 28, the pump controller 12c may decrease the pressure and return operation of the surgical pump 120 to the baseline of approximately 50 mmHg.

Typical fluid flow rates for inflow supplied by the surgical pump 120 may be approximately 50-600 ml/min. and may commonly vary from 100-400 ml/min. In response to the fluid inflow and outflow, the associated pressure measured at or inferred for the surgical site 28 may vary from approximately 30 mmHg and 80 mmHg throughout the operation of the pump 120. The baseline pressure associated with each operation or surgical procedure may be stored in the user profiles 32 for each of the associated users (e.g., surgeons, practitioners, etc.). In this way, the system 10 may identify the different operating characteristics of the pump 120 as well as the remaining device controllers of the surgical devices 12 of the system 10 for each of the user profiles 32. Similarly, the operating pressure and characteristics of the system 10 may be updated and stored to the active profile 34 of the user profiles 32 following each procedure to adjust and/or update the operating characteristics (e.g., flow rate, baseline pressure, etc.) of the system 10 based on the proposed states 210 and corresponding confirmation inputs 212 as discussed herein. In this way, the system 10 may actively store and update the operating preferences including the flow rate, baseline pressure, blood content level, and/or visibility thresholds associated with the active profile 34 data for each operation or procedure to the corresponding user profile 32. As previously discussed, the user profiles 32 may be saved to one or more servers 50 or databases for access and use in later operations of the system 10 or compatible systems with those discussed herein.

Though specifically discussed in reference to the camera apparatus 22 and the cutting or resection tool 122, the conditional mapping of the inputs of the corresponding user interfaces of the surgical devices 12 may be selectively mapped to various inputs to confirm or activate the proposed or semi-automated control settings of the surgical pump 120. For example, the control system may selectively map various controls or inputs of the surgical device 12 or peripherals in communication with the control system 10 over the device network 14. In some implementations, the system controller 24 may selectively map the user input required to confirm a proposed or semi-automated control setting to one or more surgical devices 12 that are in active operation. For example, throughout operation of the control system 10, the system controller 24 may monitor the device states associated with the device data 42 reported over the device network 14 to determine which of the surgical devices 12 is in active use or has recently been activated. In cases where a surgical device 12 has been used within a predetermined time (e.g., 10 seconds, 30 seconds, 60 seconds, etc.), the system controller 24 or the corresponding device controller 16 may conditionally map one or more inputs of the device 12 (e.g., an ablation wand, resection tool, the camera apparatus 22, etc.) to provide the confirmation of the proposed or semi-automated control of the surgical pump 120. Accordingly, the control system 10 may provide for flexible operation that may be coordinated among various surgical devices 12 to improve the accessibility of one or more of the operations identified by the vision module 60 and the state module 62 as discussed herein.

Referring now to FIGS. 9A and 9B, the operation of the vision module 60 is described in reference to an exemplary image 160 depicting the surgical site 28. In some implementations, the vision module 60 may process individual frames of the image data 26 over time and classify the frames based on identified image content in a first class 162 and a second class 164. In general, this process may be achieved via a machine learning process that implements a multi-task classifier. In operation, the multi-task classifier may implement a class activation mapping algorithm to classify the content of the image frames in one or more content classes. The classes 162, 164 may include a first content class 162 identifying a magnitude and distribution of the image data 26 in a blood-present classification 162a and a second image class indicating a no-blood-present classification 162b. Once calculated, a blood-content ratio 168 may be identified for the first content class 162 as identifying a relationship between the blood-present classification 162a and the no-blood-present classification 162b. By tracking the presence and absence of the blood content in the image data, the first content class 162 may improve an accuracy and reliability of the blood content associated with each frame of the image data 26.

In addition to the first class 162, the frames of the image data 26 may be classified in second content class 164 including a valid class 164a and an unknown class 164b. The second class 164 may indicate whether the content of the image data 26 can be classified or is valid for classification by the procedure 166 shown in FIG. 9B. The second class 164 or validity classification of the image data may be beneficial in providing an affirmative determination or classification of image frames that may not be relied upon to provide automated control with via the procedure 166. Accordingly, the multi-task classifier applied by the classification procedure may be implemented to reliably identify blood content in a video stream captured by the camera apparatus 22 and provide automated or computer-assisted control of the system 10. As demonstrated in the following example, the multi-task classifier may be implemented via class activation mapping, which may be achieved by applying an trained image classification model. In the specific example discussed, a gradient-weighted, class activation mapping procedure (e.g., GradCAM) is applied to generate the classes 162, 164.

Referring primarily to FIG. 9A, the operation of the vision module 60 used to identify the classes 162, 164 is described in reference to a range of exemplary scope images 170. The scope images 170 demonstrate a range of blood content ranging from a clear image 170a with no blood at the top of the spectrum to a blood-saturated image 170b at the bottom of the spectrum. Though not clearly visible due to the monochromatic color scale, each of the images between the clear image 170a and the blood-saturated image 170b has increasing levels of blood content and corresponding increases in red coloration. The red coloration is generally demonstrated by the increasing cloudiness moving from the top of the spectrum to the bottom. The class activation maps 172 resulting from each of the exemplary scope images 170 are discussed in reference to the image classification procedure in the example that follows.

In operation, the vision module 60 may apply the classification procedure 166 to generate a class activation map 172 identifying the blood-present classifications 162a and the no-blood-present classifications 162b for each of the processed image frames. As shown in FIG. 9A, each of the corresponding exemplary class activation maps 172 is shown for the blood-present classification 162a and the no-blood-present classification 162b aligned with the corresponding scope image 170. As shown, the scope images 170 with a lower blood content closer to the clear image 170a have high levels of class content in the no-blood-present classification 162b. In contrast, the scope images 170 with high levels of blood content closer to the blood-saturated image 170b have higher levels of content in the class activation maps 172 for the blood-present classification 162a. Based on contrast between the no-blood-present classification 162b and the blood-present classification 162a, the blood-presence ratio 168 may clearly distinguish between content in the image data that corresponds to blood and content that does not include blood. Responsive to the ratio 168 of the class activation maps 172 for the blood classifications 162a, 162b, each of the device controllers 16 may respond with one or more suggested operating states or automated operating states.

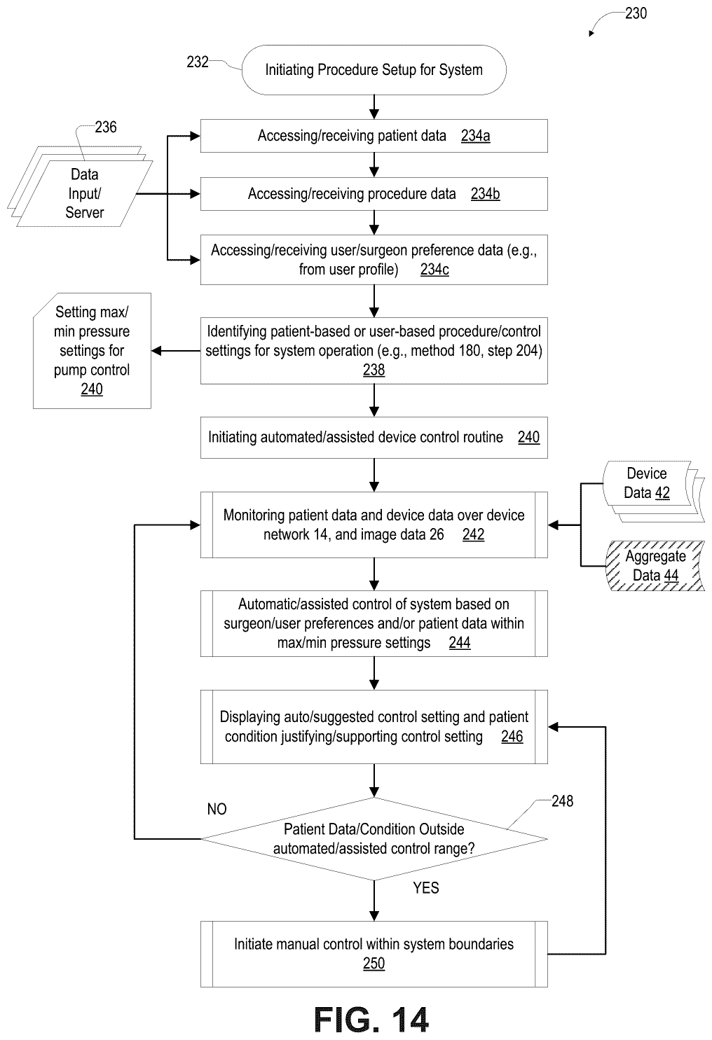

In particular reference to the operation of the arthroscopy pump controller 12c, the identification of the blood-presence ratio 168 may be applied to control a pressure of the pump 12c over a user-defined pressure range. Further details regarding the user-defined control or patient-based control of the surgical pump 120 are discussed later in reference to FIG. 14. Still referring to FIG. 9A, the operation of the pump controller 12c is described in response to the blood content demonstrated in the exemplary image 160. In the example shown, the blood content associated with the exemplary image 160 may correspond to the identified blood-content classification 174 and an identified no-blood-present classification 176. In the example shown, representative class activation maps 172 are identified for reference. As shown, the identified classifications 174, 176 may correspond to a moderate level of blood content between the clear image 170a and the blood-saturated image 170b. Based on the identified blood classifications 174, 176; the classification procedure 166 may identify a calculated blood-presence ratio 168. As shown, the calculated blood-presence ratio 168 may correspond to a moderate level of blood content as similarly identified in the classifications 174, 176. In response to the calculated blood-presence ratio 168, the pump controller 12c may automatically propose a corresponding pressure level within a user-specified range of pressure limits for a particular procedure or operation. In this way, the classification procedure 166 may be implemented to detect the blood-presence ratio 168 to accurately detect the blood content in a patient cavity and responsively control the pump pressure to remediate or control the presence of the blood in the image data 26.

Still referring to FIG. 9A, the classification procedure 166 may further identify whether each frame of the image data 26 is valid 164a or unknown 164b via the identification of the second class 164. In operation, the indication of the image data as being valid 164a may be reported over the device network to inform the device controllers 16 of the surgical devices 12 that the image data 26 is viable to support automated or semiautomated operation. Alternatively, if the image data 26 is classified as being unknown 164b for one or more image frames in series, the resulting device data 42 or aggregate data 44 may inform the device controllers 16 of the surgical devices 12 to pause or suspend semiautomatic or vision-assisted operation. In this way, the classification procedure 166 may ensure that accurate control is maintained over a variety of circumstances presented in the image data 26.

Referring now to FIG. 9B, a process diagram is shown demonstrating the classification procedure 166 in further detail. In operation, the classification procedure 166 may receive the image data 26 as a video feed comprising a plurality of image frames 166a. Each of the image frames 166a may be supplied to a neural-network or trained model comprising a plurality of layers, shown as CONV blocks and ID blocks. Each of the layers 166b may correspond to detection and processing techniques that may emphasize different objects and/or features in the image frames 166a. Once processed via the convolutional layers 166b, heat maps of the image data 26 may be combined and/or averaged via a layer combination module 166c. Following their combination, the class activation map 172 from the image frames 166a may be processed as outputs 166d in the first class 162 and the second class 164. The first class 162 or the blood/no-blood classifications 162a, 162b may be utilized to calculate the blood-presence ratio 168. Further, the determination of the valid/unknown classifications 164a, 164b may be output as a suitability indictor 166e, indicating whether the image data 26 is valid to support the automated or semiautomated operations described herein. Based on this process, the classification procedure 166 may be implemented to supply status indications identifying the blood content to each of the surgical devices 12 on the device network 14.

Referring to FIG. 10, a flow chart is shown demonstrating a method 180 for monitoring the image data and controlling the operation of the surgical pump 120. The method 180 may begin in step 182 in response to the activation of the system 10. Once activated, the system controller 24 may activate the camera apparatus 22 to capture and process the image data 26 (184). Additionally, the system controller 24 may receive and communicate various device settings and control states in the device data 42 and aggregate data 44 over the device network 14 (186). As previously discussed, the vision module 60 may include a scope location detection routine that may determine whether or not the camera apparatus 22 is inserted in the surgical site 28 (188). For example, in step 188, the vision module 60 may process the image data 26 to determine if the features and lighting associated with the image data 26 are associated with the surgical site 28 or the environment outside the surgical site (e.g., the surgical suite or operating area). One or more characteristics, for example the range of wavelengths of light, features in the image data, and a focal distance of the camera apparatus 22, may vary widely when the image data is captured in the surgical site 28 relative to the environment outside the surgical site 28 in an operating or surgical suite. Such operation may be referred to as a scope-in verses scope-out detection and may indicate whether or not the camera apparatus 22 and the corresponding image data 26 are captured depicting the surgical site 28 within the patient cavity. In response to a negative or scope-out determination of the image data 26 representing the patient cavity at the surgical site 28, the method 180 may maintain a user interface or system interface and corresponding controls presented on the display 18 in a preoperative interface mode (190) and return to step 186. If the image data 26 indicates an inserted or scope-in condition in step 188, the system controller 24 may update the controller interface to an intraoperative interface mode (192).

Responsive to the interoperative interface mode applied in step 192, the system controller 24 may control the vision module 60 to begin detecting the vision state of the surgical site 28 (194). As previously discussed, the vision state of the surgical site 28 may be detected in reference to the blood classifier 140, the visibility metric 142, and the particle-content metric 144. As shown, the method incorporates the determination of the vision state in successive steps 196, 198, and 200. In step 196, the image data 26 is determined to be blood-positive or blood-negative. In step 198, the detected particle content 168 is compared to the particle-content threshold Tₚ. Additionally, in step 200, the detected visibility 158 is compared to the visibility threshold Tᵥ. In response to the combination of each of the states identified in steps 196, 198, and 200, the vision module 60 may classify the vision state of the surgical site 28 based on the representative image data 26 in step 202. The vision state may be reported by the vision module 60 to the state module 62 and reported over the device network 14. In this way, each of the surgical devices 12 in communication over the device network 14 may update or adapt the operational settings to correspond to the detected vision state 60. Though steps 196, 198, and 200 are described in order, the detection of the clarity of the image data 26 may be rearranged based on a priority or, in some cases, processed concurrently by the vision module 60. The processing also may be undertaken periodically on a frame-by-frame basis or at a sampling frequency depending on the sophistication of the system hardware.

Following the identification and reporting of the vision state in step 202, the method 180 may proceed to step 204 where one or more of the device controllers 16 of the surgical devices 12 and/or the system controller 24 may identify a proposed control state. As discussed in reference to the pump controller 12c and the surgical pump 120, the control state may be identified based on the blood classifier 140, the visibility metric 142, and/or the particle-content metric 144. In cases where the proposed operation of the surgical pump 120 requires confirmation, the method 180 may continue to step 206 to determine if the prompt is confirmed via one or more of the inputs of the various user interfaces of the surgical devices 12 (e.g., conditionally mapped to an input or otherwise presented on a user interface of the computer or tablet 20). If confirmation is not received in step 206, the method 180 may return to step 188. If a confirmation is received, the proposed control state, in this example, of the pump controller 12c may update the operation of the surgical pump 120 to conform to the proposed control state in step 208. In this way, the control system 10 may provide for semi-automated or prompted control operations of the surgical pump 120 responsive to the vision states identified by the vision module 60.

Still referring to FIG. 10, the proposed or active control state of the surgical pump 120 may be updated by repeating or cycling steps 194-208 as previously discussed. For example, as the method 180 proceeds to step 204, the vision state identified in steps 196-200 may be updated to indicate that the image data 26 no longer includes a positive blood classification, excessive particle content, and/or a diminished visibility condition. In response to the updated vision state, the proposed control state identified in step 204 may indicate that the surgical pump 120 should be returned to a baseline pressure and typical fluid exchange rate. Accordingly, the continuing operation of the method 180 may provide for prompts associated with the activation of control states to improve the clarity or visibility of the image data 26 and also provide for operations to limit the pressure associated with the patient cavity for a surgical procedure to improve outcomes by limiting extravasation.

In some implementations, the system 10 may provide for one or more operating states and corresponding detection routines that may be step or stage-specific in relation to one or more surgical operating procedures. As previously described, the system may provide for the tracking of each stage or step of a preconfigured medical procedure based on the detection of the use of various surgical devices 12, operating characteristics of the tools reported over the device network 14, and/or corresponding conditions or features detected in the image data 26 with the procedure tracking module 64. In response to the detection of the stage or step of the procedure, the vision module 60 may identify one or more conditions, treatments, or pending operations of the system 10 that may result in changes in the clarity or visibility of the image data 26. Though some conditions may not be readily discernible based on the processing of the image data 26 alone by the image processor(s) 48, the system 10 may infer one or more states of the surgical site based on a combination of changes (e.g., coloring, cloudiness, etc.) detected in the image data 26 with the corresponding identification of the stage or step of the procedure. For example, in some steps of a procedure, the visibility of the image data 26 may be diminished due to the addition of one or more treatments or biologics to the fluid in the patient cavity at the surgical site 28. In response to such treatments, the clarity or color of the image data 26 may change; however, rinsing the cavity may limit the benefit of the treatment. Accordingly, in response to the detection of a procedural step that may include the administration of a treatment or biologic that may impact the visibility of the image data 26, the system may infer that the corresponding visibility change in the image data is the result of a procedural step (e.g., the administration of a biologic) in the patient cavity.

In response to such a detection, the controller 24 may omit or suppress a correction pump operation (e.g., rinsing or flushing the cavity) despite the apparent change in the image data 26. For example, the vision module 60 may temporarily adjust the visibility threshold Tᵥ or suppress a corresponding corrective action in response to the procedure tracking module 64 indicating that the present step in the procedure commonly includes the administration of a biologic or additive treatment that may impact the visibility of the image data 26. Similarly, the controller 24 may output a message or prompt the user of the system 10 to identify or confirm whether the change in the image quality is the result of the administration of a biologic or additive treatment to the surgical fluid. In response to a positive confirmation, the correction action of the pump controller 12c may be suppressed for a predetermined period of time or until the procedure tracking module 64 indicates that the visibility of the image data 26 should no longer be controlled based on the biologic or additive treatment. Examples of biologic treatments that may be implemented and inferred or detected by the vision module 60 may include, but are not limited, to bone marrow aspirate (BMA) concentrate, culture-expanded mesenchymal stem cells (MSCs) and stromal cells, autologous blood products (including conditioned plasma [ACP], platelet-rich plasma [PRP], etc.), growth factors, hyaluronic acid, and autologous chondrocyte implantation (ACI), autologous matrix-induced chondrogenesis (AMIC), etc.

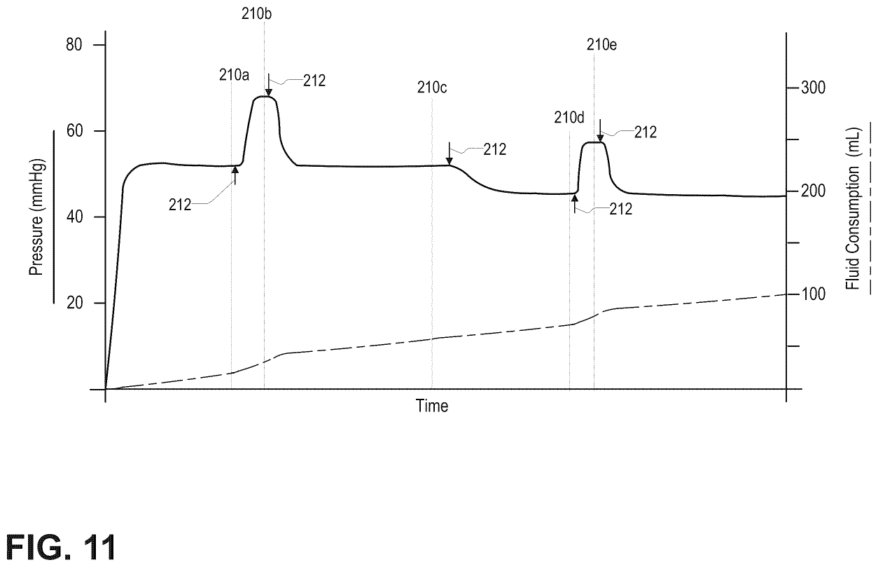

Referring now to FIG. 11, a plot is shown demonstrating the fluid pressure applied by the surgical pump 120 as well as the corresponding fluid consumption over time tracked throughout a surgical procedure. As previously discussed, in some implementations, the control system 10 (e.g., the pump controller 12c) may track the operation of one or more of the surgical devices 12, including the surgical pump 120, to determine the preferences of each user and update the preferences to the corresponding user profile 32. The data depicted in FIG. 11 may correspond to sample pump data that may be stored to identify the preferences of the associated user, such that the suggestions and the related responses may be stored in the user profile 32. In this way, the system 10 may learn the preferences associated with each of the user profiles 32 to improve the proposed operating states including the baseline pressure, fluid inflow and outflow rates, the thresholds associated with the detection of the blood classifier 140, the visibility metric 142, and the particle-content metric 144, as well as other operating settings of the devices 12 of the system 10.

In some implementations, the thresholds associated with the visibility of the surgical site 28 in the image data 26 may be identified as part of a user preference associated with each of the user profiles 32. For example, the visibility threshold Tᵥ, the particle-content threshold Tₚ, and the blood threshold associated with the blood classifier 140 each may be tracked as one or more visibility tolerance or sensitivity settings. For example, some users may prefer to flush the surgical site or apply lavage in response to a limited changes in visibility. In such cases, the system 10 may decrease one or more of the visibility threshold Tᵥ, the particle-content threshold Tₚ, and the blood threshold associated with the blood classifier 140 or increase a corresponding sensitivity for the corresponding user profile 32. In this configuration, only minor changes in visibility may trigger an automated response or prompt. Alternatively, some users may prefer only to flush the surgical site or apply lavage in response to a significant changes in visibility. In such cases, the system 10 may increase one or more of the visibility threshold Tᵥ, the particle-content threshold Tₚ, and the blood threshold associated with the blood classifier 140 or increase a corresponding sensitivity associated with the user profile 32. In this way, minor changes in visibility may not trigger an automated response or prompt, and such proposed actions may require considerable decreases in visibility before being activated. Accordingly, these user profiles 32 may be described as having a high tolerance for changes in visibility related to the one or more of the blood classifier 140, the visibility metric 142, and the particle-content metric 144.

As demonstrated in FIG. 11, the pressure readings associated with the operation of the surgical pump 120 include indications of proposed control state updates 210a, 210b, 210c, 210d, and 210e, as well as confirmation inputs 212 received by one or more of the user interfaces of the surgical devices 12. For example, at the first proposed state 210a, the vision module 60 may identify a blood-positive identification 152 from the blood classifier 140 in combination with a detected visibility 158 less than the visibility threshold Tᵥ associated with the visibility metric 142. Based on the aggregate data 44, including the reported state identified by the vision module 60 and the state module 62, the system controller 24 may output a prompt on the display 18 requesting a confirmation of an updated control state for a lavage routine. The corresponding confirmation input 212 following the first proposed state 210a may be received at the device network 14 to initiate the increased pressure and fluid exchange associated with the lavage routine. Throughout the lavage routine, the vision module 60 may monitor the image data 26 to determine if the visibility and blood content have improved to exceed the minimum visibility Tᵥ or no longer demonstrate significant blood content. Upon such a determination, the vision state may be updated by the vision module 60 and output via the state module 62 in the aggregate data 44. As shown, the second proposed state 210b is confirmed by the confirmation input 212. As demonstrated, the pressure associated with the lavage routine may be returned to the baseline pressure of approximately 50 mmHg. As previously discussed, each of the confirmation inputs 212 may be mapped to one or more inputs or user inputs associated with user interfaces of the surgical devices 12, particularly surgical devices 12 identified as being active in a predetermined time to the proposed state.

Later in the procedure, the third proposed state 210c may include a suggestion that the baseline pressure may be reduced at the surgical site 28. Such a suggestion may be identified based on reports from the vision module 60 that the image data 26 is clear of debris and blood for a period of time that may vary based on the specific procedure, patient, or user associated with the active profile 34. For example, following a predetermined time or procedure-specific period (e.g., 5-15 minutes) where the image data 26 is determined to be clear of debris (e.g., blood negative with visibility in excess of the visibility threshold Tᵥ and/or the particle-content threshold Tₚ), the controller 24 may propose or adjust an updated baseline pressure for the operation of the surgical pump 120. In response to the proposed state, the pump controller 12c may control a pressure of the inflow to the surgical site 28 to be gradually decreased to an updated baseline pressure (e.g., decreased from 52 mmHg to 44 mmHg) over an extended duration (e.g., 5-15 minutes). In this way, the system 10 may automatically propose changes to baseline pressure to improve patient recovery. Later proposed states include a fourth proposed state 210d and a fifth proposed state 210e resulting from a blood-positive identification 152 by the blood classifier 140 and a decreased visibility below the visibility threshold Tᵥ identified by the visibility metric 142, respectively. Each of the proposed control states may be identified in response to the analysis of the image data 26 processed throughout the operation allowing the users to operate the system 10 through a series of intuitive prompts that do not distract from or cause delays in the surgical procedure.

Throughout operation of the control system 10, the cumulative pressure and fluid consumption associated with the operation of the surgical pump 120 may be monitored and recorded for each procedure associated with the active profile 34 to track preferences associated with the baseline pressure, fluid exchange rate, and the increased pressure associated with the rinsing routine and/or the lavage routine. In this way, the control system 10 may track the preferences of each of the users corresponding to the user profiles 32 to improve the suggestions proposed for the operation of the surgical pump 120. Additionally, the data associated with the operation of the surgical pump 120 may be recorded in a comprehensive user library that may be compared and documented in relation to patient outcomes and pain scores to monitor the benefits associated with various pressure settings, fluid consumption rates, and various other settings associated with the operation of the pump to improve the outcomes of the patients associated with the corresponding procedures.

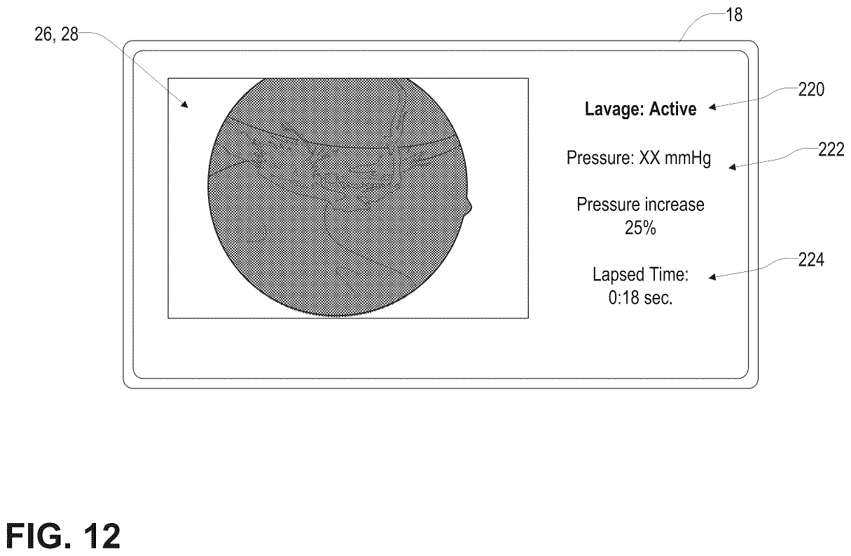

Referring now to FIG. 12, a representative diagram of a user interface is shown on the display 18. In addition to updating the control state of the surgical pump 120, the operation of the vision module 60 and the state module 62 may cause the system controller 24 to update one or more color schemes or interface menus associated with the operation of the surgical pump 120. For example, during baseline operation of the surgical pump 120, the system controller 24 may display the operating information of the control system 10 in a first color scheme (e.g., black and white). In response to the detection of an updated vision state by the vision module 60, the system controller 24 may update the color scheme to a second color scheme (e.g., green on a black background) to emphasize the identification of the proposed control state of the surgical pump 120. Additionally, in response to the confirmation of the activation of the proposed control state, the system controller 24 may update the graphic user interface and corresponding color scheme to maintain the second color scheme but focus on the updated operating information of the surgical pump 120. For example, the system controller 24 may display a lavage indication 220, as well as an associated pressure indication 222 and/or a lapsed time indication 224 associated with the lavage routine. In this way, the relevant information related to the proposed operating state activated for the surgical pump 120 may be presented and emphasized to the user of the control system 10 on the display 18.

Though discussed in reference to the active lavage setting in the corresponding color schemes, the system controller 24 may similarly activate or change the color scheme and corresponding data or information displayed on the graphic user interface to emphasize any of the proposed, confirmed, or automatically activated processes identified responsive to the vision state of the surgical site 28 determined by the vision module 60 as discussed herein.

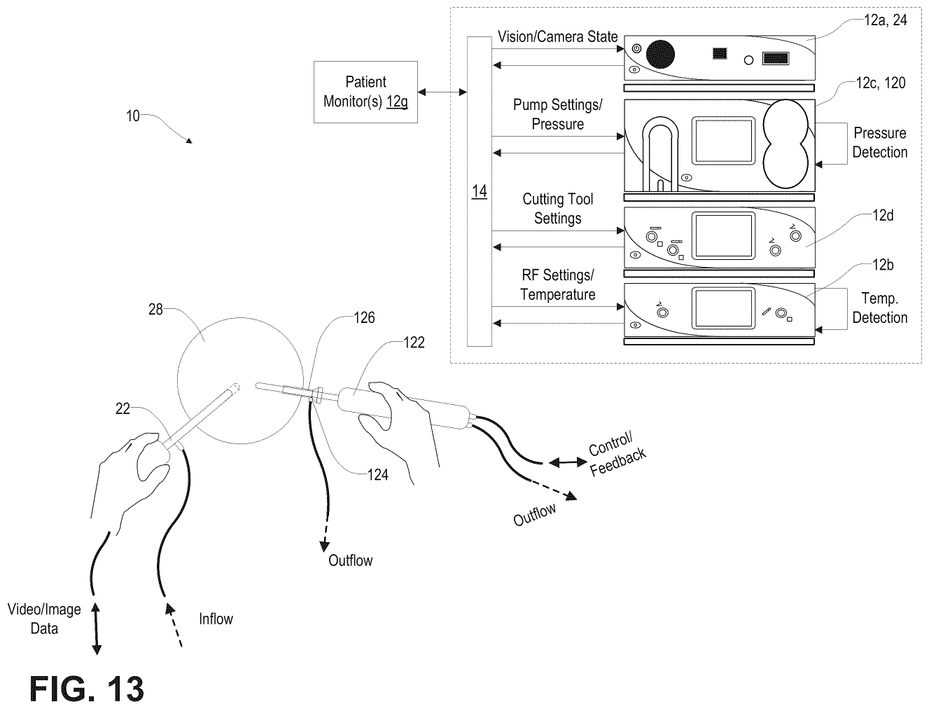

Referring now to FIG. 13, the operation of the various surgical devices 12 in communication with and/or forming the control system 10 are discussed in reference to the control of the surgical pump 120. As provided in the following examples, the operation of the surgical pump 120 may be controlled responsively to the operation of various surgical devices 12 in communication over the device network 14. For example, the control system 10 may be in communication with one or more patient monitors 12g via the device network 14. In this configuration, patient information including vital signs, such as heart rate and blood pressure, may be reported by the patient monitors 12g over the device network 14. In some implementations, the vision module 60 and/or the state module 62 may report data related to the vital signs and report corresponding information in conjunction with the states of the surgical devices 12 in the aggregate data 44 over the device network 14. In this way, the device controllers 16 of the surgical devices 12 may update or prompt the user to consider updated operational settings or operational states to conform to information reported by the patient monitors 12g.

In a particular example, an indication of the blood pressure of a patient may be monitored by the state module 62 to identify instances of increased blood pressure that may serve as lead indicators for potential bleeding at the surgical site 28. In response to the detection of elevated blood pressure by the state module 62, the pump controller 12c may prompt the user via the display 18 to increase the fluid pressure at the surgical site 28 with the surgical pump 120. As previously discussed in various examples, the user may then confirm the suggestion via an input to one or more of the user interfaces provided by the surgical devices 12. Additionally, in some cases, the operation of the vision module 60 may be updated based on the elevated blood pressure to increase the sensitivity or adjust the operational detection settings of the blood classifier 140 and/or visibility metric 142 in response to the detection of elevated blood pressure (e.g., increased diastolic, systolic, mean arterial pressure, etc.). For example, in response to the indication of elevated blood pressure, the operation of the blood classifier 140 may be prioritized or a bleeding detection threshold 154 associated with the detection of blood identified in the image data 26 at the surgical site 28 may be decreased in response to the increased likelihood of blood detection and possible hemorrhaging due to the elevated blood pressure condition. In this way, the vision module 60 may adjust the detection associated with the blood classifier 140 and/or the visibility metric 142, such that the detection associated with conditions related to elevated blood pressure may be anticipated and detected with increased sensitivity to prompt the user to apply lavage, rinse, or adjust various pump settings responsive to the condition. Examples of elevated blood pressure may correspond to systolic pressure exceeding 120 mmHg and diastolic pressure remaining less than 80 mmHg or cases where systolic pressure is elevated above 120 mmHg and diastolic pressure is greater than 80 mmHg.

In some implementations, the operation of the system 10 may similarly be updated or a user may be prompted to update the operation based on the active operation of surgical devices 12, such as the resection tool 122, an ablation or electrosurgical device, or various electromechanical surgical implements. As demonstrated in FIG. 13, operation of the resection tool 122 is demonstrated in reference to the inflow and outflow provided to the surgical site 28. As shown, outflow may be provided via both the resection tool 122 (e.g., a shaver) as well as the outflow port 124 of the cannula 126. In response to the activation of the resection tool 122, the pump controller 12c may deactivate the corresponding outflow from the resection tool 122 and increase the outflow via the outflow port 124. Such operation may ensure that the fluid pressure does not increase within the patient cavity represented by the surgical site 28 resulting from a decrease in outflow. In this way, the control system 10 may maintain homeostasis at the surgical site while also limiting potential for clogging the outflow associated with the resection tool 122.

In some implementations, the vision module 60 and state module 62 may update the settings associated with the blood classifier 140, the visibility metric 142, and/or the particle-content metric 144 based on the type of resection tool 122 implemented and/or a blade or cutting attachment style associated with the resection tool 122. For example, in some cases based on the blade or cutting attachment associated with the resection tool 122, a user may prefer to view some level of bleeding or blood presented in the image data 26 at the surgical site 28 to ensure that the procedure is effective. Under such circumstances, the style of cutting tool and/or cutting implement associated with the resection tool 122 may be reported over the device network 14 via the resection tool 122 and/or manually input into the system controller 24 during the procedural setup for the operation. In response to an indication of the cutting accessory, blade style, cutting implement, or similarly interchangeable components of the resection tool 122, the vision module 60 may update the corresponding thresholds for one or more of the blood classifier 140, the visibility metric 142, and/or the particle-content metric 144. For example, in response to the activation or connection of a rasp (e.g., power rasp), pick, power pick, or similar devices detected or identified over the device network 14, the vision module 60 may increase the minimum blood-classification threshold 154 to ensure that blood may be visible in the image data 26 without triggering automatic or prompted adjustment of the pressure or fluid flow supplied by the surgical pump 120.

As noted in the previous example, the type of resection tool, cutting accessory, blade type, proportions, aggressiveness of cut, etc. may be reported by the corresponding device controller 16 over the device network 14 as a model, serial number, tool ID, accessory ID, etc. Such information may be automatically identified by the corresponding device controller 16 as identified by one or more identification circuits (e.g., radio frequency identification circuits or conventional computerized memory devices such as electrically erasable programmable read-only memory or similar devices) of the corresponding surgical devices 12 and accessories. Additionally, the information identifying the surgical devices 12 and accessories may be manually entered preoperatively. Once loaded, each of the controller 16, 24 may identify the corresponding use over the network 14 allowing the system 10 to infer the impact on the clarity of the image data 26 and the corresponding adjustments to the operation of the vision module 60 as discussed herein.

In yet another example, the activation of the resection tool 122 may result in considerable decreases in visibility or increases in turbidity depending on the type of cutting implement or accessory associated with the resection tool 122. Accordingly, based on the style and/or type of accessory associated with the resection tool 122 (e.g., a blade, burr, pick, rasp, drill, etc.), the system controller 24 and/or the pump controller 12c may anticipate varying levels of turbidity and/or associated bleeding. In such cases, the control system 10 may adjust the associated pressure and suction corresponding to the inflow and outflow associated with the operation of the surgical pump 120 based on the style, model, type, proportions, or other variables that may change in relation to the specific resection tool 122 implemented. For example, in some cases, the pressure adjustment associated with the inflow of the surgical fluid may be adjusted from approximately 5% to 50% or more elevated from baseline pressure in relation to the detection of decreased visibility by the vision module 60 depending on the style, model, type, etc. of the accessory associated with the resection tool 122 and the likely resulting impact on the visibility at the surgical site 28. Similarly, the system controller 24 and/or pump controller 12c may adjust one or more rinse settings that adjust the fluid exchange rate supplied by the inflow and returned via the outflow to clear the visibility related to the associated operation of a resection tool 122. For example, the pump controller 12c may increase the fluid exchange rate of the surgical fluid supplied to the surgical site 28 from approximately 5% to 50% or more depending on the anticipated resulting turbidity or particle content associated with the operation of the specific model or style of the resection tool 122 implemented. By tracking the model and type of resection tool 122 implemented, the control system 10 may flexibly respond to the anticipated level of turbidity, particle content, and/or likely blood content at the surgical site 28 and automatically initiate or suggest customized pressure or fluid exchange settings associated with the operation of the surgical pump 120 in combination with the specific resection tool 122 to limit unnecessary pressure at the surgical site 28 as well as customize or tailor the response of the surgical pump 120 to the specific conditions anticipated for the surgical devices 12 implemented.

In some cases, the control system 10 may additionally adjust the operation of the surgical pump 120 to assist in maintaining optimal performance for one or more of the surgical devices 12. For example, in response to the operation of an electrosurgical or ablation probe, the electrosurgical console 12b may communicate the corresponding operating configuration, model, style, etc. of the electrosurgical device over the device network 14. In response to the indication of the electrosurgical probe (e.g., an RF ablation probe), the pump controller 12c may respond by decreasing a fluid exchange rate (e.g., decrease fluid transmission or exchange by 5%-25% or more) of the surgical fluid to the surgical site 28 associated with the inflow and outflow. By limiting the supply and/or suction rates associated with the operation of surgical pump 120, the pump controller 12c may ensure that excessive suction does not impact the generation of the plasma filed generated by the ablation probe.

In some implementations, a temperature of the surgical site 28 may be detected by the ablation probe, camera apparatus 22, or other surgical devices 12 and reported over the device network 14. In response to elevated levels of temperature, the suction or inflow and outflow controlled by the surgical pump 120 may increase the fluid exchange rate at the surgical site 28 in response to elevated levels of temperature that may result from the operation of the ablation probe. In this way, the control system 10 may decrease or adjust a baseline pressure and associated fluid exchange rate in response to the utilization of the ablation probe as controlled by the electrosurgical console 12b and selectively increase the fluid exchange at the surgical site 28 on-demand to avoid temperature increases above one or more predetermined temperature thresholds in the patient cavity. Such operation may ensure that the performance of the ablation probe is maintained while also limiting temperature increases in the patient cavity.

As provided in the various foregoing examples, the control system 10 may provide for a variety of benefits in relation to the prompting of suggested controls and/or automatic activation of various control settings of the surgical devices 12 and particularly the surgical pump 120 to improve the effective operation of the system 10. By combining the distributed controls provided over the device network with the operation of the vision module 60 and the state module 62, the control system 10 may monitor the operation of each of the associated surgical devices 12 in relation to the activity and/or characteristics that may be associated with or identified within the patient cavity at the surgical site 28 to ensure that each of the corresponding elements or devices 12 of the control system 10 may effectively operate in concert. Though specific examples of the combined operations are provided throughout the application, it shall be understood that each of the control routines and coordinated operations of the surgical devices 12 may be applied individually or in combination without departing from the inventive concepts disclosed herein.

Referring now to FIG. 14, a flow chart is shown demonstrating a method 230 implemented to set up or adjust operating parameters for one or more of the surgical devices 12 in communication with the control system 10. In general, the method 230 may be implemented to set user-specified pressure limits or guide rails associated with the operation of the surgical pump 120, which may be associated with the user profile 32. In addition to the user-specified limits, the pressure settings for the surgical pump 120 may be adjusted based on one or more preexisting conditions or patient health characteristics that may be entered by staff and/or accessed by a patient database in communication with the system 10. In some cases, the pressure settings associated with the operation of the pump 120 may further be controlled in response to patient information, including vital signs, such as heart rate and blood pressure, that may be reported by the patient monitors 12g during the procedure. Accordingly, the method 230 may be flexibly implemented to adjust a maximum pressure setting that may be automatically applied to control bleeding or hemorrhaging and a minimum pressure setting that may be maintained between bleeding events as a baseline pressure for operating the surgical pump 120.

As demonstrated in FIG. 14, the method 230 may begin by initiating a set-up procedure for the control system 10 (232). The set-up procedure may include one or more configuration steps 234a, 234b, 234c that may include accessing or receiving data from a user-input device, database, and/or server 236. As previously described, the configuration data may be received in the form of patient data 234a, procedure data 234b, and/or user/surgeon preference data 234c that may be accessed via the user-profile 32. The patient data 234a may correspond to one or more preexisting conditions, for example, irregular blood pressure (e.g., hypertension, hypotension), heart disease, diabetes, age, atrophy, or other patient health characteristics. Additionally, the patient data 234a in step 234 may correspond to interoperative patient data reported by the patient monitors 12g over the device network 14. The pressure settings for the surgical pump 120 may additionally be set based on a specific type of procedure, as indicated in step 234b. Also, as demonstrated in step 234c, the maximum and minimum pressure settings for the surgical pump 120 may be adjusted based on a user or surgeon preference. Though each of the data inputs associated with the operation of the system 10 are described separately in steps 234a, 234b, 234c; it shall be understood that the user or surgeon preference data may incorporate a maximum and minimum pressure setting for specific procedures as outlined in step 234b as well as based on various preexisting health conditions associated with each patient as describe in step 234a. Accordingly, the set up of the system 10 for the semiautomated operation of the surgical pump 120 may be customized to suit the specific needs of the patient, the procedure, and/or the user/surgeon.

Once the data associated with steps 234a, 234b, and 234c is accessed by the device controller 16 of the surgical pump 120, the controller 16 may identify the control settings based on one or more of the associated parameters identified in steps 234a, 234b, 234c to adjust the control settings for operating the system 10, particularly the surgical pump 120 (238). Based on each of the input parameters from steps 234a, 234b, 234c, the device controller 16 may set a maximum pressure setting and a minimum pressure setting, between which the pressure of the surgical pump 120 may be controlled automatically or by proposed setting updates. In this way, the operation of the surgical pump 120 or other devices 12 may be adjusted in response to the vision states described in reference to FIGS. 6-9 (240). With the patient, user and/or procedure-based pressure settings identified in step 238 and recorded in step 240, the automated control procedures for the surgical pump 120 may be initiated in step 240.

The automated or assisted device control routine 240 may operate as generally outlined in reference to the method 180. Alternatively, the pressure control of the surgical pump 120 between the maximum and minimum pressure settings may be adjusted progressively in response to the blood-presence ratio 168 or other indicators that blood is present in the image data 26. For example, rather than adjusting the pressure of the surgical pump 120 responsive to the particulate content or visibility being below a predetermined threshold, the device controller 16 of the surgical pump 120 may gradually adjust (increase, decrease) the pump pressure in response to increases or decreases in the blood-presence ratio 168. To effectively provide for suggested or automated updates to the pressure setting of the surgical pump 120, the control routine 240 may continue by monitoring the patient data reported by the patient monitors 12g and may update the pressure settings and/or limits defined in step 238 (242). In step 242, the routine 240 may also process and/or classify the image data 26 to identify the vision state recorded by the camera apparatus 22. As previously discussed, the device data 42 and image data 26 may be reported over the device network 14 as individual device data and/or aggregate data 44, which may be compiled by the system controller 24 or camera control units (CCUs).

With the patient data, device data 42 and/or aggregate data 44, the device controller 16 may automatically adjust the pressure of the surgical pump 120 responsive to each of the data inputs reported in step 242 (244). With the automated or assisted pump pressure control settings identified in step 244, the routine 240 may continue by displaying the control settings to the user on the one or more displays 18 (246). When displaying the automated and/or suggested control setting for the pump 120, the device controller 16 may additionally identify a justification or reason for the pressure setting, which may be associated with the patient data or procedure data associated with steps 234a, 234b and/or the patient and device data monitored in step 242. In this way, the method 230 may provide beneficial justifications for proposed or automated pressure setting updates that may be detected based on conditions in the image data before users may even identify a bleeding event has occurred.

Throughout operation of the system 10, the control routine 240 may monitor the patient data and corresponding conditions presented in the image data to determine if the automated or proposed pressure settings are within the maximum and minimum pressure settings set in step 240 (248). For example, during a bleeding event, if a patient begins to hemorrhage and the image data reports a high blood-presence ratio 168, the device controller 16 may be adjusted to the maximum pressure setting associated with the user preference. However, under such circumstances, a user may be prompted to initiate a manual control that may allow the pressure to exceed the user-specified maximum and minimum pressure settings and extend to a maximum or minimum pressure setting that may be associated with the limit set by the device manufacturer (250). Under such circumstances, the automated or assisted device control routine 240 may be at least temporarily disabled, allowing the user or surgeon to adjust the pressure settings within the maximum and minimum limits set by the manufacturer of the surgical pump 120. In this way, the control routine 240 may identify conditions that may require manual intervention to adjust the pressure settings of the pump 120 to provide the best care for the patient. Alternatively, in step 248, if the automated or assisted pump pressure settings remain within the user-specified maximum and minimum pressure settings, the routine 240 may continue back to step 242 to monitor and maintain the visibility presented in the image data 26.

Though the method 230 is described as primarily providing for control of the pressure of the pump 120 responsive to the image data 26, the pressure and/or inflow and outflow of the surgical pump 120 may similarly be adjusted or controlled responsive to the detected operation of one or more of the surgical devices 12 reported over the device network 14 as described in step 242. For example, the activation of a resection or electrosurgical device reported in the device data 42 and/or aggregate data 44 may prompt the device controller 16 of the surgical pump 120 to increase an inflow and outflow through the surgical site to improve the visibility of the image data 26. Such indications of the operation of the devices 12 may be applied in combination with the various detection and classification routines described in reference to FIGS. 6-9 to optimize the response of the system 10 to various control scenarios. By automating or proposing control settings based on the image data 26 and device data reported over the device network 14, the system 10 may improve the operation of the surgical pump 120 in coordination with each of the devices communicating over the device network 14. Further, by adjusting the control or pressure settings of the pump 120 based on the patient data, procedure data, and/or the user/surgeon preferences, the disclosure may provide for a customized user experience that may improve operation of the surgical pump 120 to improve surgical outcomes.

According to some aspects of the disclosure, a surgical control system for a plurality of surgical devices comprises at least one device controller in communication with each of the plurality of surgical devices via a communication bus. The at least one device controller configured to communicate device data comprising messages indicative of at least one of a control state and a detected condition associated with the operation of each of the plurality of surgical devices. A camera apparatus is in communication with the plurality of medical devices via the communication bus, the camera apparatus comprising at least one controller configured to capture image data demonstrating a surgical site; process the image data; identify a surgery status of the surgical site in response to the image data; generate aggregate data comprising the surgery status and the device data in a packet communicated over the communication bus; and communicate the aggregate data over the communication bus. Each of the plurality of device controllers independently controls an operation of the plurality of surgical devices in response to at least one of the device data and the aggregate data.

According to various aspects, the disclosure may implement one or more of the following features or configurations in various combinations:
the surgery status is identified in response to a combination of the device data with the image data and comprises an indication of at least one of a patient condition, a procedural step, a surgical device condition, and a surgical site condition;
the device data is associated with the operation of each of the plurality of medical devices and comprises messages communicating at least one of a control state and a detected condition monitored by one or more of the plurality of medical devices;
the detected condition is detected by at least one of the surgical devices and identifies a state of the surgical site;
the detected condition comprises at least one of temperature data, pressure data (pressure of a fluid in a cavity), flow rate data, and current data (feedback);
the detected condition is an operating condition of one or more of the plurality of surgical devices;
the detected condition is a patient condition (e.g., patient vitals, health statistics) monitored by a patient monitor of the plurality of surgical devices;
at least one device controller comprises a plurality of device controllers associated with each of the plurality of surgical devices;
each of the device controllers independently reports and monitors the device data communicated as messages from each of the plurality of surgical devices;
the plurality of surgical devices independently respond selectively to the aggregate data packet or the device data in accordance with a programming control structure of a corresponding device controller of the plurality of device controllers;
at least one controller processes the device data as a factor in the determination of the surgery status;
the factor associated with the device data directs the at least one controller to identify the surgery status within a subset of a plurality of status categories;
at least one controller infers a current surgery status in response to the device data;
at least one controller infers the current surgery status further in response to a previously identified surgery status, such that the surgery status is identified in response to a combination of the device data and the previously identified surgery status; and/or
at least one controller is configured to identify a proposed control configuration for at least one of the surgical devices based on the aggregate data.

According to another aspect of the disclosure, a method for controlling a surgical control system in communication with a plurality of surgical devices comprises broadcasting device data including messages indicative of at least one of a control state and a detected condition associated with the operation of each of the plurality of surgical devices via a communication bus in communication with a plurality of device controllers of the plurality of surgical devices; capturing image data demonstrating a surgical site with a camera apparatus; identifying a surgery status of the surgical site in response to the image data; generating aggregate data comprising the surgery status and the device data in a packet; and communicating the aggregate data over the communication bus, wherein each of the plurality of device controllers independently controls an operation of the plurality of surgical devices in response to at least one of the device data and the aggregate data.

According to various aspects, the disclosure may implement one or more of the following features or configurations in various combinations:
the device data is reported by the each of the plurality of medical devices and comprises messages communicating at least one of a control state and a detected condition monitored by one or more of the plurality of medical devices;
the detected condition is detected by at least one of the surgical devices and identifies a state of the surgical site;
identifying a proposed control configuration for at least one of the surgical devices based on the aggregate data;
displaying the proposed control configuration as a prompt requesting a confirmation of the proposed control configuration;
mapping an input of a user interface of an active medical device of the plurality of medical devices or the camera apparatus, wherein the input of the user interface is mapped to receive a confirmation of the proposed control configuration in response to an activation of the input; and/or
in response to the input to the user interface associated with the confirmation, the user interface communicates the confirmation of the proposed control configuration to the communication bus.

According to yet another aspect of the disclosure, a surgical control system for a plurality of surgical devices comprises a plurality of device controllers in communication with each of the plurality of surgical devices via a communication bus, wherein each of the device controllers independently reports the device data from one of the plurality of surgical devices and monitors the device data communicated as messages from each of the plurality of surgical devices wherein the device data is indicative of at least one of a control state and a detected condition associated with the operation of each of the plurality of surgical devices; a camera apparatus in communication with the plurality of medical devices via the communication bus, the camera apparatus comprising at least one controller configured to capture image data demonstrating a surgical site and identify a surgery status of the surgical site in response to the image data, wherein the surgery status is reported by the camera apparatus over the communication bus; and wherein each of the plurality of device controllers independently controls an operation of the plurality of surgical devices in response to at least one of the device data and the surgery status.

It will be understood that any described processes or steps within described processes may be combined with other disclosed processes or steps to form structures within the scope of the present device. The exemplary structures and processes disclosed herein are for illustrative purposes and are not to be construed as limiting.

It is also to be understood that variations and modifications can be made on the aforementioned structures and methods without departing from the concepts of the present device, and further it is to be understood that such concepts are intended to be covered by the following claims unless these claims by their language expressly state otherwise.

The above description is considered that of the illustrated embodiments only. Modifications of the device will occur to those skilled in the art and to those who make or use the device. Therefore, it is understood that the embodiments shown in the drawings and described above are merely for illustrative purposes and not intended to limit the scope of the device, which is defined by the following claims as interpreted according to the principles of patent law, including the Doctrine of Equivalents

1. A surgical control system for a plurality of surgical devices comprising:
at least one device controller in communication with each of the plurality of surgical devices via a communication bus, the at least one device controller configured to communicate device data comprising messages indicative of at least one of a control state and a detected condition associated with the operation of each of the plurality of surgical devices;
a camera apparatus in communication with the plurality of medical devices via the communication bus, the camera apparatus comprising at least one controller configured to:
capture image data demonstrating a surgical site;
process the image data;
identify a surgery status of the surgical site in response to the image data;
generate aggregate data comprising the surgery status and the device data in a packet communicated over the communication bus; and
communicate the aggregate data over the communication bus; and
wherein each of the plurality of device controllers independently controls an operation of the plurality of surgical devices in response to at least one of the device data and the aggregate data.

2. The surgical control system according to claim 1, wherein the surgery status is identified in response to a combination of the device data with the image data and comprises an indication of at least one of a patient condition, a procedural step, a surgical device condition, and a surgical site condition.

3. The surgical control system according to claim 1, wherein the device data is associated with the operation of each of the plurality of medical devices and comprises messages communicating at least one of a control state and a detected condition monitored by one or more of the plurality of medical devices.

4. The surgical control system according to claim 3, wherein the detected condition is detected by at least one of the surgical devices and identifies a state of the surgical site.

5. The surgical control system according to claim 4, wherein the detected condition comprises at least one of temperature data, pressure data, flow rate data, and current data.

6. The surgical control system according to claim 3, wherein the detected condition is an operating condition of one or more of the plurality of surgical devices.

7. The surgical control system according to claim 3, wherein the detected condition is a patient condition monitored by a patient monitor of the plurality of surgical devices.

8. The surgical control system according to claim 1, wherein the at least one device controller comprises a plurality of device controllers associated with each of the plurality of surgical devices.

9. The surgical control system according to claim 8, wherein each of the device controllers independently reports and monitors the device data communicated as messages from each of the plurality of surgical devices.

10. The surgical control system according to claim 1, wherein the plurality of surgical devices independently respond selectively to the aggregate data packet or the device data in accordance with a programming control structure of a corresponding device controller of the plurality of device controllers.

11. The surgical control system according to claim 1, wherein the at least one controller processes the device data as a factor in the determination of the surgery status.

12. The surgical control system according to claim 11, wherein the factor associated with the device data directs the at least one controller to identify the surgery status within a subset of a plurality of status categories.

13. The surgical control system according to claim 1, wherein the at least one controller infers a current surgery status in response to the device data.

14. The surgical control system according to claim 13, wherein the at least one controller infers the current surgery status further in response to a previously identified surgery status, such that the surgery status is identified in response to a combination of the device data and the previously identified surgery status.

15. The surgical control system according to claim 1, wherein the at least one controller is configured to identify a proposed control configuration for at least one of the surgical devices based on the aggregate data.

16. A method for controlling a surgical control system in communication with a plurality of surgical devices, the method comprising:
broadcasting device data comprising messages indicative of at least one of a control state and a detected condition associated with the operation of each of the plurality of surgical devices via a communication bus in communication with a plurality of device controllers of the plurality of surgical devices;
capturing image data demonstrating a surgical site with a camera apparatus;
identifying a surgery status of the surgical site in response to the image data;
generating aggregate data comprising the surgery status and the device data in a packet; and
communicating the aggregate data over the communication bus, wherein each of the plurality of device controllers independently controls an operation of the plurality of surgical devices in response to at least one of the device data and the aggregate data.

17. The method according to claim 16, wherein the device data is reported by the each of the plurality of medical devices and comprises messages communicating at least one of a control state and a detected condition monitored by one or more of the plurality of medical devices.

18. The surgical control system according to claim 17, wherein the detected condition is detected by at least one of the surgical devices and identifies a state of the surgical site.

19. The method according to claim 16, further comprising:
identifying a proposed control configuration for at least one of the surgical devices based on the aggregate data; and
displaying the proposed control configuration as a prompt requesting a confirmation of the proposed control configuration.

20. The method according to claim 19, further comprising:
mapping an input of a user interface of an active medical device of the plurality of medical devices or the camera apparatus, wherein the input of the user interface is mapped to receive a confirmation of the proposed control configuration in response to an activation of the input.

21. The method according to claim 19, wherein in response to the input to the user interface associated with the confirmation, the user interface communicates the confirmation of the proposed control configuration to the communication bus.

22. A surgical control system for a plurality of surgical devices comprising:
a plurality of device controllers in communication with each of the plurality of surgical devices via a communication bus, wherein each of the device controllers independently reports the device data from one of the plurality of surgical devices and monitors the device data communicated as messages from each of the plurality of surgical devices wherein the device data is indicative of at least one of a control state and a detected condition associated with the operation of each of the plurality of surgical devices;
a camera apparatus in communication with the plurality of medical devices via the communication bus, the camera apparatus comprising at least one controller configured to capture image data demonstrating a surgical site and identify a surgery status of the surgical site in response to the image data, wherein the surgery status is reported by the camera apparatus over the communication bus; and
wherein each of the plurality of device controllers independently controls an operation of the plurality of surgical devices in response to at least one of the device data and the surgery status.
